SEQUENCE LISTING (64 sequences)

SEQ ID NO: 1 - protein, 6 aa, synthetic construct
  REGION 1..6: note = -
NNYVMC

SEQ ID NO: 2 - protein, 17 aa, synthetic construct
  REGION 1..17: note = -
CINTGSGSTYYATWAKG

SEQ ID NO: 3 - protein, 21 aa, synthetic construct
  REGION 1..21: note = -
STTYYNYIGGNWIYVMDGFNL

SEQ ID NO: 4 - protein, 11 aa, synthetic construct
  REGION 1..11: note = -
QASENIYSSLA

SEQ ID NO: 5 - protein, 7 aa, synthetic construct
  REGION 1..7: note = -
DASDLAS

SEQ ID NO: 6 - protein, 12 aa, synthetic construct
  REGION 1..12: note = -
QQGYTVDNIDNT

SEQ ID NO: 7 - protein, 29 aa, synthetic construct
  REGION 1..29: note = -
QSLEESGGDLVKPGASLTLTCTASGFSFS

SEQ ID NO: 8 - protein, 14 aa, synthetic construct
  REGION 1..14: note = -
WVRQAPGKGLEWIG

SEQ ID NO: 9 - protein, 31 aa, synthetic construct
  REGION 1..31: note = -
RFTISKISSTTVTLQMTSLTAADTATYFCAR

SEQ ID NO: 10 - protein, 11 aa, synthetic construct
  REGION 1..11: note = -
WGPGTLVPVSS

SEQ ID NO: 11 - protein, 23 aa, synthetic construct
  REGION 1..23: note = -
AQVLTQTPSSVSEPVGGTVTINC

SEQ ID NO: 12 - protein, 15 aa, synthetic construct
  REGION 1..15: note = -
WYQQKPGQPPKLLIY

SEQ ID NO: 13 - protein, 32 aa, synthetic construct
  REGION 1..32: note = -
GVPSRFSGSGSGTEYTLTISGVECDDAATYYC

SEQ ID NO: 14 - protein, 10 aa, synthetic construct
  REGION 1..10: note = -
FGGGTEVVVK

SEQ ID NO: 15 - protein, 129 aa, synthetic construct
  REGION 1..129: note = -
QSLEESGGDLVKPGASLTLTCTASGFSFSNNYVMCWVRQAPGKGLEWIGCINTGSGSTYYATWAKGRFTISKISSTTVTLQMTSLTAADTATYFCARSTTYYNYIGGNWIYVMDGFNLWGPGTLVPVSS

SEQ ID NO: 16 - protein, 110 aa, synthetic construct
  REGION 1..110: note = -
AQVLTQTPSSVSEPVGGTVTINCQASENIYSSLAWYQQKPGQPPKLLIYDASDLASGVPSRFSGSGSGTEYTLTISGVECDDAATYYCQQGYTVDNIDNTFGGGTEVVVK

SEQ ID NO: 17 - protein, 6 aa, synthetic construct
  REGION 1..6: note = -
TYYFMC

SEQ ID NO: 18 - protein, 17 aa, synthetic construct
  REGION 1..17: note = -
CIATSSGSTWYANWVNG

SEQ ID NO: 19 - protein, 14 aa, synthetic construct
  REGION 1..14: note = -
WDVSYAGDGYGFNL

SEQ ID NO: 20 - protein, 11 aa, synthetic construct
  REGION 1..11: note = -
QASQSISNDLN

SEQ ID NO: 21 - protein, 7 aa, synthetic construct
  REGION 1..7: note = -
KASTLAS

SEQ ID NO: 22 - protein, 12 aa, synthetic construct
  REGION 1..12: note = -
QQGYSSSNVDNV

SEQ ID NO: 23 - protein, 30 aa, synthetic construct
  REGION 1..30: note = -
QEQLVESGGGLVQPEGSLTLTCKGSGFDLS

SEQ ID NO: 24 - protein, 14 aa, synthetic construct
  REGION 1..14: note = -
WVRQAPGKGLEWIG

SEQ ID NO: 25 - protein, 31 aa, synthetic construct
  REGION 1..31: note = -
RFSISKTSSTTVTLQMTSLTAADTATYFCAR

SEQ ID NO: 26 - protein, 11 aa, synthetic construct
  REGION 1..11: note = -
WGPGTLVTVSS

SEQ ID NO: 27 - protein, 22 aa, synthetic construct
  REGION 1..22: note = -
YDMTQTPSSVSAAVGGTVTINC

SEQ ID NO: 28 - protein, 15 aa, synthetic construct
  REGION 1..15: note = -
WYQQKPGQPPKLLTY

SEQ ID NO: 29 - protein, 32 aa, synthetic construct
  REGION 1..32: note = -
GVPSRFKGSGSGTQFTLTISGIECADAATYYC

SEQ ID NO: 30 - protein, 10 aa, synthetic construct
  REGION 1..10: note = -
FGGGTEVVVK

SEQ ID NO: 31 - protein, 123 aa, synthetic construct
  REGION 1..123: note = -
QEQLVESGGGLVQPEGSLTLTCKGSGFDLSTYYFMCWVRQAPGKGLEWIGCIATSSGSTWYANWVNGRFSISKTSSTTVTLQMTSLTAADTATYFCARWDVSYAGDGYGFNLWGPGTLVTVSS

SEQ ID NO: 32 - protein, 109 aa, synthetic construct
  REGION 1..109: note = -
YDMTQTPSSVSAAVGGTVTINCQASQSISNDLNWYQQKPGQPPKLLTYKASTLASGVPSRFKGSGSGTQFTLTISGIECADAATYYCQQGYSSSNVDNVFGGGTEVVVK

SEQ ID NO: 33 - protein, 5 aa, synthetic construct
  REGION 1..5: note = -
TYSMG

SEQ ID NO: 34 - protein, 16 aa, synthetic construct
  REGION 1..16: note = -
IINTGGGAYYASWAKG

SEQ ID NO: 35 - protein, 10 aa, synthetic construct
  REGION 1..10: note = -
ESLIYGGFHI

SEQ ID NO: 36 - protein, 11 aa, synthetic construct
  REGION 1..11: note = -
QASQSISNALA

SEQ ID NO: 37 - protein, 7 aa, synthetic construct
  REGION 1..7: note = -
GASNLAS

SEQ ID NO: 38 - protein, 13 aa, synthetic construct
  REGION 1..13: note = -
QSTYYGSSYVGGA

SEQ ID NO: 39 - protein, 29 aa, synthetic construct
  REGION 1..29: note = -
QSVEESGGRLVTPGTPLTLTCTVSGIDLS

SEQ ID NO: 40 - protein, 14 aa, synthetic construct
  REGION 1..14: note = -
WVRQAPGKGLEYIG

SEQ ID NO: 41 - protein, 30 aa, synthetic construct
  REGION 1..30: note = -
RFTISRTSTTVDLKITSPTTEDTATYFCAR

SEQ ID NO: 42 - protein, 11 aa, synthetic construct
  REGION 1..11: note = -
WGPGTLVTVSL

SEQ ID NO: 43 - protein, 23 aa, synthetic construct
  REGION 1..23: note = -
DVVMTQTPASVSEPVGGTVTIKC

SEQ ID NO: 44 - protein, 15 aa, synthetic construct
  REGION 1..15: note = -
WYQQKPGQRPNLLIY

SEQ ID NO: 45 - protein, 32 aa, synthetic construct
  REGION 1..32: note = -
GVPSRFTGSRSGTEFTLTISDLECADAATYYC

SEQ ID NO: 46 - protein, 10 aa, synthetic construct
  REGION 1..10: note = -
FGGGTEVVVK

SEQ ID NO: 47 - protein, 115 aa, synthetic construct
  REGION 1..115: note = -
QSVEESGGRLVTPGTPLTLTCTVSGIDLSTYSMGWVRQAPGKGLEYIGIINTGGGAYYASWAKGRFTISRTSTTVDLKITSPTTEDTATYFCARESLIYGGFHIWGPGTLVTVSL

SEQ ID NO: 48 - protein, 111 aa, synthetic construct
  REGION 1..111: note = -
DVVMTQTPASVSEPVGGTVTIKCQASQSISNALAWYQQKPGQRPNLLIYGASNLASGVPSRFTGSRSGTEFTLTISDLECADAATYYCQSTYYGSSYVGGAFGGGTEVVVK

SEQ ID NO: 49 - protein, 5 aa, synthetic construct
  REGION 1..5: note = -
SYYMI

SEQ ID NO: 50 - protein, 16 aa, synthetic construct
  REGION 1..16: note = -
FINTGGGAYYASWAKG

SEQ ID NO: 51 - protein, 13 aa, synthetic construct
  REGION 1..13: note = -
GGAPDVNDYGYDI

SEQ ID NO: 52 - protein, 11 aa, synthetic construct
  REGION 1..11: note = -
QASQNIVGRLA

SEQ ID NO: 53 - protein, 7 aa, synthetic construct
  REGION 1..7: note = -
GASTLAS

SEQ ID NO: 54 - protein, 14 aa, synthetic construct
  REGION 1..14: note = -
QSNYGADSTTYGVV

SEQ ID NO: 55 - protein, 29 aa, synthetic construct
  REGION 1..29: note = -
QSVEESGGRLVKPDESLTLTCTASGFSLS

SEQ ID NO: 56 - protein, 14 aa, synthetic construct
  REGION 1..14: note = -
WVRQAPGKGLECIG

SEQ ID NO: 57 - protein, 30 aa, synthetic construct
  REGION 1..30: note = -
RFTISRTSTTVDLKMTSLTTEDTATYFCAR

SEQ ID NO: 58 - protein, 11 aa, synthetic construct
  REGION 1..11: note = -
WGPGTLVTVSL

SEQ ID NO: 59 - protein, 23 aa, synthetic construct
  REGION 1..23: note = -
DIVMTQTPASVSEPVGGTVTIKC

SEQ ID NO: 60 - protein, 15 aa, synthetic construct
  REGION 1..15: note = -
WYQQKPGQPPKLLIY

SEQ ID NO: 61 - protein, 32 aa, synthetic construct
  REGION 1..32: note = -
GVPSRFKGSGSGTQFTLTISDLECDDAATYYC

SEQ ID NO: 62 - protein, 10 aa, synthetic construct
  REGION 1..10: note = -
FGGGTEVVVR

SEQ ID NO: 63 - protein, 118 aa, synthetic construct
  REGION 1..118: note = -
QSVEESGGRLVKPDESLTLTCTASGFSLSSYYMIWVRQAPGKGLECIGFINTGGGAYYASWAKGRFTISRTSTTVDLKMTSLTTEDTATYFCARGGAPDVNDYGYDIWGPGTLVTVSL

SEQ ID NO: 64 - protein, 112 aa, synthetic construct
  REGION 1..112: note = -
DIVMTQTPASVSEPVGGTVTIKCQASQNIVGRLAWYQQKPGQPPKLLIYGASTLASGVPSRFKGSGSGTQFTLTISDLECDDAATYYCQSNYGADSTTYGVVFGGGTEVVVR

(19) United States
(12) Patent Application Publication
(10) Pub. No.: US 2025/0042976 A1
(43) Pub. Date: Feb. 6, 2025

(54) MONOCLONAL ANTIBODIES SPECIFIC FOR SARS-COV-2 RBD

(71) Applicant: Roche Diagnostics Operations, Inc., Indianapolis, IN (US)

(72) Inventors: Michael Gerg, Muenchen (DE); Simon Jochum, Muenchen (DE); Ute Jucknischke, Iffeldorf (DE); Ulrike Kurtkaya, Graefelfing (DE); Michael Schraeml, Penzberg (DE); Sandrine Carolina Stiegler, Zürich (CH)

(21) Appl. No.: 18/704,654

(22) PCT Filed: Oct. 25, 2022
(86) PCT No.: PCT/EP2022/079718

§ 371 Date: Apr. 25, 2024

(30) Foreign Application Priority Data
Oct. 26, 2021 (EP) 21204850.8

(51) Int. Cl. C07K 16/10

(52) U.S. Cl. CPC C07K 16/1003; C07K 2317/565; C07K 2317/567; C07K 2317/76; C07K 2317/92; C07K 2317/94

(57) ABSTRACT

The present invention relates to monoclonal antibodies binding to the Receptor Binding Domain of the Spike protein of SARS-CoV-2 virus, nucleic acids encoding said antibody, host cells producing the same, compositions and kits comprising said antibodies, method of detecting SARS-CoV-2 virus in a sample comprising using said antibodies and methods of using said antibodies in immunoassays.

The present invention relates to monoclonal antibodies binding to the receptor binding domain (RBD) of the spike protein of SARS-CoV-2 virus, nucleic acids encoding said antibody, host cells producing the same, compositions and kits comprising said antibodies, methods of detecting said SARS-CoV-2 viruses in a sample comprising using said antibodies and methods of using said antibodies in immunoassays.

### BACKGROUND OF THE INVENTION

Coronaviruses (CoVs) are large, enveloped, positive-sense, single-stranded RNA viruses and based on their serological and genotypic characters, they can be further subdivided into Alpha-, Beta-, Gamma- and Deltacoronaviruses. The two Betacoronaviruses SARS-CoV-1 (severe acute respiratory syndrome coronavirus) and MERS-CoV (Middle East respiratory syndrome coronavirus) have caused two severe coronaviral epidemics in the past decade (SARS 2002/2003, MERS 2012). During December 2019, an outbreak of a novel infectious respiratory disease termed Coronavirus Disease 2019 (COVID-19) emerged in China and became a global pandemic by March 2020. Since 31 Dec. 2019 and as of Jul. 5, 2021 184,677,763 reported cases with 3,995,429 confirmed deaths have been reported worldwide with 221 countries or territories being affected (source World Health Organization—https://www.who.int/emergencies/diseases/novel-coronavirus-2019). COVID-19 is caused by a novel coronavirus, the severe acute respiratory syndrome coronavirus 2 (SARS-CoV-2). SARS-CoV-2 infects the respiratory tract by binding of the host cell receptor ACE2 (angiotensin-converting enzyme 2), a receptor that is also widely present in the lower respiratory tract. The surface spike (S) glycoprotein of SARS-CoV-2 mediates this interaction with the ACE2 receptor, drives membrane fusion and therefore host cell entry. The spike protein (S) is a trimeric protein and the main target for vaccines and inhibitors of viral entry (Walls et al, 2020).

Common symptoms of COVID-19 include fever, cough, fatigue, shortness of breath or breathing difficulties. These symptoms are relatively non-specific and can be seen in a variety of other diseases. While most COVID-19 patients have mild symptoms, some develop pneumonia, acute respiratory distress syndrome, septic shock, and kidney failure.

The burden of COVID-19 extends far beyond that of a contagious disease and threatens to overwhelm healthcare systems. It will be crucial to identify where the disease burden is high for ensuring prudent and effective distribution of emergency medical care and public health resources. The risk of severe outcomes associated with COVID-19 seems to increase with age, frailty and vascular comorbidities. This scenario is thought to increase hospitalization, intensive care unit admission, and hospital readmissions. Since SARS-CoV-2 is a novel virus, experience in patient management from diagnosis to therapy and vaccination is lacking.

The standard method of testing for a SARS-CoV-2 infection is real-time reverse transcriptase polymerase chain reaction (real-time RT-PCR), of nasopharyngeal and oropharyngeal swab samples from patients. However, molecular testing is rather slow and expensive and cannot offer testing the magnitude that is required to respond to the COVID-19 pandemic. The demand for PCR-based SARS-CoV-2 tests is high and the supply is still problematic as the pandemic continues.

Antibody Tests, like anti-nucleocapsid or anti-spike Immunoassays followed the PCR testings in the laboratory setting to assess immunity of patients. Antigen tests close the gap between molecular testing (PCR) and immunity testing (antibody test).

Rapid antigen tests were developed in a Point of Care set up aiming to respond to the high demand of testing and to allow for SARS-CoV-2 infection as early as possible. However, there is no antigen test for the central laboratory setting on the market, which allows for high throughput testing to increase SARS-CoV-2 testing capacity worldwide. In view of the ongoing pandemic and increase in infected patients and thus, demand for testing, there is a high demand for cost efficient and high-throughput antigen testing in a centralized lab set up. Such fully automated systems can provide test results in 18 minutes for a single test (excluding time for sample collection, transport, and preparation), with a throughput of up to 300 tests per hour from a single analyser, depending on the analyser. A laboratory based automated antigen assay allows for cost and error reduction due to removal of manual handling as well as fast turn-around times and high test throughput.

### SUMMARY OF THE INVENTION

In a first aspect, the present invention relates to an (isolated) monoclonal antibody or antigen-binding fragment thereof that binds to the Receptor Binding Domain (RBD) of the Spike protein of SARS-CoV-2 virus
a) with an association rate constant (kₐ) of more than 2.5E+06 M⁻¹s⁻¹, as determined by surface plasmon resonance,
and/or
b) with a dissociation rate constant (k_{d}) of less than 5.0E-03 s⁻¹, as determined by surface plasmon resonance,
and/or
c) with a half-life time of t/2diss of 4 minutes or more, as determined by surface plasmon resonance,
and/or
d) with a 1:1 or 1:2 stoichiometry.

In a preferred embodiment of the first aspect of the present invention, the antibody is neutralizing.

In a preferred embodiment of the first aspect of the present invention, the antibody is inhibitory.

In a second aspect, the present invention relates to an isolated antibody or an antigen-binding fragment thereof, which
a) comprises CDR-H1, CDR-H2, CDR-H3, CDR-L1, CDR-L2 and CDR-L3 according to SEQ ID NO: 1, 2, 3, 4, 5, and 6, respectively,
b) binds to the same epitope as an antibody comprising CDR-H1, CDR-H2, CDR-H3, CDR-L1, CDR-L2 and CDR-L3 according to SEQ ID NO: 1, 2, 3, 4, 5, and 6, respectively,
   or
c) competes for binding to the RBD of the spike protein of SARS-CoV-2 virus with an antibody comprising CDR-H1, CDR-H2, CDR-H3, CDR-L1, CDR-L2 and CDR-L3 according to SEQ ID NO: 1, 2, 3, 4, 0 5, and 6, respectively.

In a third aspect, the present invention relates to an isolated antibody or an antigen-binding fragment thereof, which
a) comprises CDR-H1, CDR-H2, CDR-H3, CDR-L1, CDR-L2 and CDR-L3 according to SEQ ID NO: 17, 18, 19, 20, 21, and 22, respectively,
b) binds to the same epitope as an antibody comprising CDR-H1, CDR-H2, CDR-H3, CDR-L1, CDR-L2 and CDR-L3 according to SEQ ID NO: 17, 18, 19, 20, 21, and 22, respectively,
or
c) which competes for binding to the RBD of the spike protein of SARS-CoV-2 virus with an antibody comprising CDR-H1, CDR-H2, CDR-H3, CDR-L1, CDR-L2 and CDR-L3 according to SEQ ID NO: 17, 18, 19, 20, 21 and 22, respectively.

In a fourth aspect, the present invention relates to an isolated antibody or an antigen-binding fragment thereof, which
a) comprises CDR-H1, CDR-H2, CDR-H3, CDR-L1, CDR-L2 and CDR-L3 according to SEQ ID NO: 33, 34, 35, 36, 37, and 38, respectively,
b) binds to the same epitope as an antibody comprising CDR-H1, CDR-H2, CDR-H3, CDR-L1, CDR-L2 and CDR-L3 according to SEQ ID NO: 33, 34, 35, 36, 37, and 38, respectively,
or
c) which competes for binding to the RBD of the spike protein of SARS-CoV-2 virus with an antibody comprising CDR-H1, CDR-H2, CDR-H3, CDR-L1, CDR-L2 and CDR-L3 according to SEQ ID NO: 33, 34, 35, 36, 37, and 38, respectively.

In a fifth aspect, the present invention relates to an isolated antibody or an antigen-binding fragment thereof, which:
a) comprises CDR-H1, CDR-H2, CDR-H3, CDR-L1, CDR-L2 and CDR-L3 according to SEQ ID NO: 49, 50, 51, 52, 53 and 54, respectively,
b) binds to the same epitope as an antibody comprising CDR-H1, CDR-H2, CDR-H3, CDR-L1, CDR-L2 and CDR-L3 according to SEQ ID NO: 49, 50, 51, 52, 53 and 54, respectively,
or
c) which competes for binding to the RBD of the spike protein of SARS-CoV-2 virus with an antibody comprising CDR-H1, CDR-H2, CDR-H3, CDR-L1, CDR-L2 and CDR-L3 according to SEQ ID NO: 49, 50, 51, 52, 53 and 54, respectively.

In preferred embodiments, the antibodies according to the second, third, fourth and fifth aspects of the present invention, are neutralizing antibodies.

In preferred embodiments, the antibodies according to the second, third, fourth and fifth aspect of the present invention, are inhibitory antibodies.

In a sixth aspect, the present invention relates to a kit comprising at least one antibody selected from the group of antibodies as described above for the first aspect, the second aspect, the third aspect, the fourth aspect, or the fifth aspect of the present invention, and optionally a second antibody selected from the group of antibodies as described above for the first aspect, the second aspect, the third aspect, the fourth aspect, or the fifth aspect of the present invention, and optionally a third antibody selected from the group of antibodies as described above for the first aspect, the second aspect, the third aspect, the fourth aspect, or the fifth aspect of the present invention.

In a seventh aspect, the present invention relates to a nucleic acid encoding an antibody selected from the group of antibodies as described above for the first aspect, the second aspect, the third aspect, the fourth aspect, or the fifth aspect of the present invention.

In an eighth aspect, the present invention relates to a host cell comprising the nucleic acid as described above for the seventh aspect of the present invention, and/or producing an antibody as described above for the first aspect, the second aspect, the third aspect, the fourth aspect, or the fifth aspect of the present invention.

In a ninth aspect, the present invention relates to a composition comprising at least one antibody selected from the group of antibodies as described above for the first aspect, the second aspect, the third aspect, the fourth aspect, or the fifth aspect of the present invention.

In a tenth aspect, the present invention relates to the use of an antibody of the first aspect, the second aspect, the third aspect, the fourth aspect or the fifth aspect of the present invention, or the kit of the sixth aspect of the present invention or the composition of the ninth aspect of the present invention, for an in vitro immunoassay.

In an eleventh aspect, the present invention relates to an in vitro method for detecting the presence of SARS-CoV-2 virus in a sample obtained from a patient.

The embodiments according to the present invention will be described in more detail below.

### LIST OF THE FIGURES

FIG. 1: Kinetic Screening with exemplary kinetic signatures of antibody/RBD (wildtype) interactions. (A) Deselected after Screening. (B) Further recommended after Screening.

FIG. 2: Kinetic constants for clones 1F12, 4H10, 7G5 and 14F10.

FIG. 3: Antibody interactions with the RBD (wildtype) with increasing RBD concentrations from 0.2 nM to 13.3 nM in presence of BSA. Shown are concentration series with a duplicate for concentration 13.3 nM (black) overlayed by a Langmuir 1:1 binding model, Rₘₐₓ global, RI=0 (grey).

FIG. 4: Exemplary sensorgram overlays for epitope binning experiments on complex formation of in house wildtype RBD with antibody pairs. Grey arrows indicate the start (up) and stop (down) of the injections 1) primary antibody, 2) blocking mixture, 3) in house RBD, 4) primary antibody again, 5) secondary antibody, 6) regeneration. A) Clone 2C11 as primary antibody and 7G5 as secondary antibody (upper sensorgram) form an immune complex with RBD. The lower sensorgram shows a negative control with 2C11 as primary and secondary antibody. No positive response is detectable in the negative control run in time section 5. B) Zoom—Clone 2C11 as primary and 7G5 as secondary antibody (upper sensorgram) vs. negative controls (i) 2C11 respectively buffer as secondary antibody (ii) buffer instead of secondary antibody and buffer instead of RBD and 2C11 as secondary antibody (lower sensorgram). No positive response is detectable for both negative control runs in time section 5. C) Zoom—7G5 as primary antibody and 2C11 as secondary antibody (upper sensorgram), forming a sandwich complex with RBD, respectively negative control 7G5 as primary and secondary antibody and 7G5 as primary and secondary primary antibody and buffer instead of a RBD (lower sensorgrams). Combining the information from various experiments, 21 epitope regions were identified.

FIG. 5: Epitope binning determination: the molar ratios of 9×9 antibodies are summarized in the matrix. Antibody 4H10 is shown as representative antibody in an epitope binning matrix consisting of nine tested antibodies. Here, 81 antibody pairing combinations were analyzed.

FIG. 6: Identification of ACE-2 RBD interface binders.

Two consecutive injections (1) 1st wildtype RBD and (2) 2nd ACE2 are offered to the surface displayed anti-RBD mAb clones 4H10, 1F12, 1H9 and 2C11. (3) ACE-2 injection stop and dissociation phase. A) No ACE-2 response signal increase nor decrease in the binary complex dissociation phases [2-3] shown with clones 4H10 and 1F12. These clones bind to the wildtype RBD in or close by the ACE-2/RBD interface and completely avoid the ACE-2/RBD docking. B) ACE-2 increasing response signal and ternary mAb/RBD/ACE-2 complex formation shown with clones 1H9 and 2C11. These clones bind to the wildtype RBD remotely from the ACE-2/RBD interface.

FIG. 7: Assessing interference of ACE2-RBD binding by the antibodies on an Elecsys® platform by a competitive immunoassay. This data confirm the BIACORE® data.

FIG. 8: Kinetic profiles for mAb 1F12 binding a selection of RBD-mutants at 37° C. obtained via SPR. Antibody-interactions with increasing concentrations of RBD mutants, c=1.2-33.3 nM resp. 3.7-33.3 nM overlaid with the Langmuir 1:1 binding model.

FIG. 9: Comparison between RBD wildtype vs. mutants with regards to affinity, association rate constants kₐ and complex stability (t_{/2 diss}) (binding of clone 1F12 to different mutants (variants) of SARS-CoV-2).

FIG. 10: Relative affinity RBD wildtype vs. mutants (binding of clone 1F12 to different mutants (variants) of SARS-CoV-2).

FIG. 11: Relative association rate constants kₐ RBD and complex stability (t_{/2 diss}) wildtype vs. mutants (binding of clone 1F12 to different mutants (variants) of SARS-CoV-2).

FIG. 12: Kinetic constants for the binding of clone 1F12 to the wildtype and to mutants (variants) of SARS-CoV-2. Row 1 shows the results for the wildtype. Row 2 for mutant SARS-CoV-2-RBD-N501Y; row 3 for mutant SARS-CoV-2 RBD-E484K; row 4 for mutant SARS-CoV-2 RBD-E484K N501Y; row 5 for mutant SARS-CoV-2 RBD-Q-His8_L452R, E484Q; row 6 for mutant SARS-CoV-2 RBD-Q-His8 L452R, N501Y; row 7 for mutant SARS-CoV-2 RBD-Q-His8 E406W; row 8 for mutant SARS-CoV-2 RBD-Q-His8 K417T, E484K, N501Y; row 9 for mutant SARS-CoV-2 RBD-Q-His8 K417N, E484K, N501Y; row 10 for mutant SARS-CoV-2 Omicron; row 11 for mutant SARS-CoV-2 BA2; row 12 for mutant SARS-CoV-2 BA2.12.1; row 13 for mutant SARS-CoV-2 BA2.11; row 14 for mutant SARS-CoV-2 RBD Q L452R, T478K; row 15 for mutant SARS-CoV-2 RBD Q L452Q F490S; row 16 for mutant SARS-CoV-2 RBD Q R346K E484K N501Y; row 17 for mutant SARS-CoV-2 RBD Q_BA5

List of the Sequences

SEQ ID NO: 1 Antibody 1F12: CDR-H1: NNYVMC
SEQ ID NO: 2 Antibody 1F12: CDR-H2: CINTGSGSTYYATWAKG
SEQ ID NO: 3 Antibody 1F12: CDR-H3: STTYYNYIGGNWIYVMDGFNL
SEQ ID NO: 4 Antibody 1F12: CDR-L1: QASENIYSSLA
SEQ ID NO: 5 Antibody 1F12: CDR-L2: DASDLAS
SEQ ID NO: 6 Antibody 1F12: CDR-L3: QQGYTVDNIDNT
SEQ ID NO: 7 Antibody 1F12: FR-H1:
QSLEESGGDLVKPGASLTLTCTASGFSFS
SEQ ID NO: 8 Antibody 1F12: FR-H2: WVRQAPGKGLEWIG
SEQ ID NO: 9 Antibody 1F12: FR-H3:
RFTISKISSTTVTLQMTSLTAADTATYFCAR
SEQ ID NO: 10 Antibody 1F12: FR-H4: WGPGTLVPVSS
SEQ ID NO: 11 Antibody 1F12: FR-L1: AQVLTQTPSSVSEPVGGTVTINC
SEQ ID NO: 12 Antibody 1F12: FR-L2: WYQQKPGQPPKLLIY
SEQ ID NO: 13 Antibody 1F12: FR-L3:
GVPSRFSGSGSGTEYTLTISGVECDDAATYYC
SEQ ID NO: 14 Antibody 1F12: FR-L4: FGGGTEVVVK
SEQ ID NO: 15 Antibody 1F12: heavy chain variable domain:
QSLEESGGDLVKPGASLTLTCTASGFSFSNNYVMCWVRQ
APGKGLEWIGCINTGSGSTYYATWAKGRFTISKISSTTVTL
QMTSLTAADTATYFCARSTTYYNYIGGNWIYVMDGENL
WGPGTLVPVSS
SEQ ID NO: 16 Antibody 1F12: light chain variable domain:
AQVLTQTPSSVSEPVGGTVTINCQASENIYSSLAWYQQKP
GQPPKLLIYDASDLASGVPSRFSGSGSGTEYTLTISGVECD
DAATYYCQQGYTVDNIDNTFGGGTEVVVK
SEQ ID NO: 17 Antibody 4H10: CDR-H1: TYYFMC
SEQ ID NO: 18 Antibody 4H10: CDR-H2: CIATSSGSTWYANWVNG
SEQ ID NO: 19 Antibody 4H10: CDR-H3: WDVSYAGDGYGENL
SEQ ID NO: 20 Antibody 4H10: CDR-L1: QASQSISNDLN
SEQ ID NO: 21 Antibody 4H10: CDR-L2: KASTLAS
SEQ ID NO: 22 Antibody 4H10: CDR-L3: QQGYSSSNVDNV
SEQ ID NO: 23 Antibody 4H10: FR-H1:
QEQLVESGGGLVQPEGSLTLTCKGSGFDLS
SEQ ID NO: 24 Antibody 4H10: FR-H2: WVRQAPGKGLEWIG
SEQ ID NO: 25 Antibody 4H10: FR-H3:
RFSISKTSSTTVTLQMTSLTAADTATYFCAR
SEQ ID NO: 26 Antibody 4H10: FR-H4: WGPGTLVTVSS
SEQ ID NO: 27 Antibody 4H10: FR-L1: YDMTQTPSSVSAAVGGTVTINC
SEQ ID NO: 28 Antibody 4H10: FR-L2: WYQQKPGQPPKLLTY
SEQ ID NO: 29 Antibody 4H10: FR-L3:
GVPSRFKGSGSGTQFTLTISGIECADAATYYC
SEQ ID NO: 30 Antibody 4H10: FR-L4: FGGGTEVVVK
SEQ ID NO: 31 Antibody 4H10: heavy chain variable domain:
QEQLVESGGGLVQPEGSLTLTCKGSGFDLSTYYFMCWVR
QAPGKGLEWIGCIATSSGSTWYANWVNGRFSISKTSSTTV
TLQMTSLTAADTATYFCARWDVSYAGDGYGFNLWGPGT
LVTVSS
SEQ ID NO: 32 Antibody 4H10: light chain variable domain:
YDMTQTPSSVSAAVGGTVTINCQASQSISNDLNWYQQKP
GQPPKLLTYKASTLASGVPSRFKGSGSGTQFTLTISGIECA
DAATYYCQQGYSSSNVDNVFGGGTEVVVK
SEQ ID NO: 33 Antibody 7G5: CDR-H1: TYSMG
SEQ ID NO: 34 Antibody 7G5: CDR-H2: IINTGGGAYYASWAKG
SEQ ID NO: 35 Antibody 7G5: CDR-H3: ESLIYGGFHI
SEQ ID NO: 36 Antibody 7G5: CDR-L1: QASQSISNALA
SEQ ID NO: 37 Antibody 7G5: CDR-L2: GASNLAS
SEQ ID NO: 38 Antibody 7G5: CDR-L3: QSTYYGSSYVGGA
SEQ ID NO: 39 Antibody 7G5: FR-H1:
QSVEESGGRLVTPGTPLTLTCTVSGIDLS
SEQ ID NO: 40 Antibody 7G5: FR-H2: WVRQAPGKGLEYIG
SEQ ID NO: 41 Antibody 7G5: FR-H3:
RFTISRTSTTVDLKITSPTTEDTATYFCAR
SEQ ID NO: 42 Antibody 7G5: FR-H4: WGPGTLVTVSL
SEQ ID NO: 43 Antibody 7G5: FR-L1: DVVMTQTPASVSEPVGGTVTIKC
SEQ ID NO: 44 Antibody 7G5: FR-L2: WYQQKPGQRPNLLIY
SEQ ID NO: 45 Antibody 7G5: FR-L3:
GVPSRFTGSRSGTEFTLTISDLECADAATYYC
SEQ ID NO: 46 Antibody 7G5: FR-L4: FGGGTEVVVK
SEQ ID NO: 47 Antibody 7G5: heavy chain variable domain:
QSVEESGGRLVTPGTPLTLTCTVSGIDLSTYSMGWVRQAP
GKGLEYIGIINTGGGAYYASWAKGRFTISRTSTTVDLKITS
PTTEDTATYFCARESLIYGGFHIWGPGTLVTVSL
SEQ ID NO: 48 Antibody 7G5: light chain variable domain:
DVVMTQTPASVSEPVGGTVTIKCQASQSISNALAWYQQKP
GQRPNLLIYGASNLASGVPSRFTGSRSGTEFTLTISDLECAD
AATYYCQSTYYGSSYVGGAFGGGTEVVVK
SEQ ID NO: 49 Antibody 14F10: CDR-H1: SYYMI
SEQ ID NO: 50 Antibody 14F10: CDR-H2: FINTGGGAYYASWAKG
SEQ ID NO: 51 Antibody 14F10: CDR-H3: GGAPDVNDYGYDI
SEQ ID NO: 52 Antibody 14F10: CDR-L1: QASQNIVGRLA
SEQ ID NO: 53 Antibody 14F10: CDR-L2: GASTLAS
SEQ ID NO: 54 Antibody 14F10: CDR-L3: QSNYGADSTTYGVV
SEQ ID NO: 55 Antibody 14F10: FR-H1:
QSVEESGGRLVKPDESLTLTCTASGFSLS
SEQ ID NO: 56 Antibody 14F10: FR-H2: WVRQAPGKGLECIG
SEQ ID NO: 57 Antibody 14F10: FR-H3:
RFTISRTSTTVDLKMTSLTTEDTATYFCAR
SEQ ID NO: 58 Antibody 14F10: FR-H4: WGPGTLVTVSL
SEQ ID NO: 59 Antibody 14F10: FR-L1: DIVMTQTPASVSEPVGGTVTIKC
SEQ ID NO: 60 Antibody 14F10: FR-L2: WYQQKPGQPPKLLIY
SEQ ID NO: 61 Antibody 14F10: FR-L3:
GVPSRFKGSGSGTQFTLTISDLECDDAATYYC
SEQ ID NO: 62 Antibody 14F10: FR-L4: FGGGTEVVVR
SEQ ID NO: 63 Antibody 14F10: heavy chain variable domain:
QSVEESGGRLVKPDESLTLTCTASGFSLSSYYMIWVRQAP
GKGLECIGFINTGGGAYYASWAKGRFTISRTSTTVDLKMT
SLTTEDTATYFCARGGAPDVNDYGYDIWGPGTLVTVSL
SEQ ID NO: 64 Antibody 14F10: light chain variable domain:
DIVMTQTPASVSEPVGGTVTIKCQASQNIVGRLAWYQQKP
GQPPKLLIYGASTLASGVPSRFKGSGSGTQFTLTISDLECD
DAATYYCQSNYGADSTTYGVVFGGGTEVVVR

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The various examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “150 mg to 600 mg” should be interpreted to include not only the explicitly recited values of 150 mg to 600 mg, but to also include individual values and sub-ranges within the indicated range. Thus, included in this numerical range are individual values such as 150, 160, 170, 180, 190, . . . 580, 590, 600 mg and sub-ranges such as from 150 to 200, 150 to 250, 250 to 300, 350 to 600, etc. This same principle applies to ranges reciting only one numerical value. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

“Symptoms” of a disease are implication of the disease noticeable by the tissue, organ or organism having such disease and include but are not limited to pain, weakness, tenderness, strain, stiffness, and spasm of the tissue, an organ or an individual. “Signs” or “signals” of a disease include but are not limited to the change or alteration such as the presence, absence, increase or elevation, decrease or decline, of specific indicators such as biomarkers or molecular markers, or the development, presence, or worsening of symptoms. Symptoms of pain include, but are not limited to an unpleasant sensation that may be felt as a persistent or varying burning, throbbing, itching or stinging ache.

The term “disease” and “disorder” are used interchangeably herein, referring to an abnormal condition, especially an abnormal medical condition such as an illness or injury, wherein a tissue, an organ or an individual is not able to efficiently fulfil its function anymore. Typically, but not necessarily, a disease is associated with specific symptoms or signs indicating the presence of such disease. The presence of such symptoms or signs may thus, be indicative for a tissue, an organ or an individual suffering from a disease. An alteration of these symptoms or signs may be indicative for the progression of such a disease. A progression of a disease is typically characterised by an increase or decrease of such symptoms or signs which may indicate a “worsening” or “bettering” of the disease. The “worsening” of a disease is characterised by a decreasing ability of a tissue, organ or organism to fulfil its function efficiently, whereas the “bettering” of a disease is typically characterised by an increase in the ability of a tissue, an organ or an individual to fulfil its function efficiently. A tissue, an organ or an individual being at “risk of developing” a disease is in a healthy state but shows potential of a disease emerging. Typically, the risk of developing a disease is associated with early or weak signs or symptoms of such disease. In such case, the onset of the disease may still be prevented by treatment. Examples of a disease include but are not limited to infectious diseases, traumatic diseases, inflammatory diseases, cutaneous conditions, endocrine diseases, intestinal diseases, neurological disorders, joint diseases, genetic disorders, autoimmune diseases, and various types of cancer.

The term “Coronaviruses” refers to a group of related viruses that cause diseases in mammals and birds. In humans, Coronaviruses cause respiratory tract infections that can range from mild to lethal. Mild illnesses include some cases of the common cold, while more lethal varieties can cause “SARS”, “MERS”, and “COVID-19”. Coronaviruses contain a positive-sense, single-stranded RNA genome.

The viral envelope is formed by a lipid bilayer wherein the membrane (M), envelope (E) and spike (S) structural proteins are anchored. Inside the envelope, multiple copies of the nucleocapsid (N) protein form the nucleocapsid, which is bound to the positive-sense single-stranded RNA genome in a continuous beads-on-a-string type conformation. Its genome comprises Orfs 1a and 1b encoding the replicase/transcriptase polyprotein, followed by sequences encoding the spike (S)-envelope protein, the envelope (E)-protein, the membrane (M)-protein and the nucleocapsid (N)-protein. Interspersed between these reading frames are the reading frames for the accessory proteins, which differ between the different virus strains.

Several human Coronaviruses are known, four of which lead to rather mild symptoms in patients:
Human Coronavirus NL63 (HCoV-NL63), α-CoV
Human Coronavirus 229E (HCoV-229E), α-CoV
Human Coronavirus HKU1 (HCoV-HKU1), β-CoV
Human Coronavirus OC43 (HCoV-OC43), β-CoV
HCoV-NL63, HCoV-229E, HCoV-HKU1, and HCoV-OC43 are often referred to as “common cold coronaviruses”.

Three human Coronaviruses produce symptoms that are potentially severe:
Middle East respiratory syndrome-related Coronavirus (MERS-CoV), β-CoV
Severe acute respiratory syndrome Coronavirus (SARS-CoV), β-CoV
Severe acute respiratory syndrome Coronavirus 2 (SARS-CoV-2), β-CoV

SARS-Cov-2 causes Coronavirus disease 2019 (COVID-19). Because the strain was first discovered in Wuhan, China, it is sometimes referred to as the Wuhan virus. In the context of the present application, this strain is referred to as the wildtype strain. Several mutants of the wildtype strain have appeared since the first discovery of the virus. These mutant strains are well known to the skilled person and are well described in the prior art (see for example: https://www.cdc.gov/coronavirus/2019-ncov/variants/variant-classifications.html). In the context of the present application, the term SARS-CoV-2 refers to the wildtype strain as well to the mutant strains (also known as variants). SARS-Cov-2 is highly contagious in humans, and the World Health Organization (WHO) has designated the still ongoing pandemic of COVID-19 a Public Health Emergency of International Concern. The earliest case of infection currently known is thought to have been found on 17 Nov. 2019. The SARS-Cov-2 sequence was first published on Jan. 10, 2020 (Wuhan-Hu-1, GenBank accession number MN908947). Subsequent to the first outbreak in Wuhan, the virus spread to all provinces of China and to more than 150 other countries in Asia, Europe, North America, South America, Africa, and Oceania. Symptoms include high-fever, sore throat, dry cough, and exhaustion. In severe cases, pneumonia may develop.

The term “natural Coronavirus” refers to a coronavirus as occurring in nature, i.e. to any coronavirus as disclosed above, both wildtype strains as also mutant strains (variants). It is understood that a natural Coronavirus comprises all proteins and nucleic acid molecules present in a naturally occurring virus. In difference to a natural Coronavirus, “viral fragments”, “virus-like particles”, or Corona specific antigens, only comprise some but not all proteins and nucleic acid molecules present in a naturally occurring virus. Accordingly, such “viral fragments”, “virus-like particles”, or Coronavirus-specific antigens are not infectious but are still able to inflict an immune response in a patient. Accordingly, vaccination with Coronavirus-specific viral fragments, Coronavirus-specific virus-like particles, or Coronavirus-specific antigens inflicts the productions of antibodies against those viral fragments, virus-like particles, or antigens, in the patient.

The term “measurement”, “measuring”, “detecting”, “detection”, “determining” or “determination” comprises a qualitative, a semi-quanitative or a quantitative measurement. The term “detecting the presence” refers to a qualitative measurement, indicating the presence of absence without any statement to the quantities (e.g. yes or no statement). The term “detecting amount” refers to a quantitative measurement wherein the absolute number is detected (ng). The term “detecting the concentration” refers to a quantitative measurement wherein the amount is determined in relation to a given volume (e.g. ng/ml).

As used herein, a “patient” means any mammal, fish, reptile or bird that may benefit from the determination or diagnosis described herein. In particular, a “patient” is selected from the group consisting of laboratory animals (e.g. mouse, rat, rabbit, or zebrafish), domestic animals (including e.g. guinea pig, rabbit, horse, donkey, cow, sheep, goat, pig, chicken, camel, cat, dog, turtle, tortoise, snake, lizard or goldfish), or primates including chimpanzees, bonobos, gorillas and human beings. It is particularly preferred that the “patient” is a human being.

The term “sample” or “sample of interest” are used interchangeably herein, referring to a part or piece of a tissue, organ or individual, typically being smaller than such tissue, organ or individual, intended to represent the whole of the tissue, organ or individual. Upon analysis a sample provides information about the tissue status or the health or diseased status of an organ or individual. Examples of samples include but are not limited to fluid samples such as nasopharyngeal swabs, oropharyngeal swabs, blood, serum, plasma, synovial fluid, urine, saliva, and lymphatic fluid, or solid samples such as tissue extracts, cartilage, bone, synovium, and connective tissue. Analysis of a sample may be accomplished on a visual or chemical basis. Visual analysis includes but is not limited to microscopic imaging or radiographic scanning of a tissue, organ or individual allowing for morphological evaluation of a sample. Chemical analysis includes but is not limited to the detection of the presence or absence of specific indicators or alterations in their amount or level.

The term “host cell” refers to a cell that harbours a vector (e.g. a plasmid or virus). Such host cell may either be a prokaryotic (e.g. a bacterial cell) or a eukaryotic cell (e.g. a fungal, plant or animal cell). Host cells include both single-cellular prokaryote and eukaryote organisms (e.g., bacteria, yeast, and actinomycetes) as well as single cells from higher order plants or animals when being grown in cell culture.

The term “amino acid” generally refers to any monomer unit that comprises a substituted or unsubstituted amino group, a substituted or unsubstituted carboxy group, and one or more side chains or groups, or analogs of any of these groups. Exemplary side chains include, e.g., thiol, seleno, sulfonyl, alkyl, aryl, acyl, keto, azido, hydroxyl, hydrazine, cyano, halo, hydrazide, alkenyl, alkynl, ether, borate, boronate, phospho, phosphono, phosphine, heterocyclic, enone, imine, aldehyde, ester, thioacid, hydroxylamine, or any combination of these groups. Other representative amino acids include, but are not limited to, amino acids comprising photoactivatable cross-linkers, metal binding amino acids, spin-labeled amino acids, fluorescent amino acids, metal-containing amino acids, amino acids with novel functional groups, amino acids that covalently or noncovalently interact with other molecules, photocaged and/or photoisomerizable amino acids, radioactive amino acids, amino acids comprising biotin or a biotin analog, glycosylated amino acids, other carbohydrate modified amino acids, amino acids comprising polyethylene glycol or polyether, heavy atom substituted amino acids, chemically cleavable and/or photocleavable amino acids, carbon-linked sugar-containing amino acids, redox-active amino acids, amino thioacid containing amino acids, and amino acids comprising one or more toxic moieties. As used herein, the term “amino acid” includes the following twenty natural or genetically encoded alpha-amino acids: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). In cases where “X” residues are undefined, these should be defined as “any amino acid.” The structures of these twenty natural amino acids are shown in, e.g., Stryer et al., Biochemistry, 5th ed., Freeman and Company (2002). Additional amino acids, such as selenocysteine and pyrrolysine, can also be genetically coded for (Stadtman (1996) “Selenocysteine,” Annu Rev Biochem. 65:83-100 and Ibba et al. (2002) “Genetic code: introducing pyrrolysine,” Curr Biol. 12(13):R464-R466). The term “amino acid” also includes unnatural amino acids, modified amino acids (e.g., having modified side chains and/or backbones), and amino acid analogs. See, e.g., Zhang et al. (2004) “Selective incorporation of 5-hydroxytryptophan into proteins in mammalian cells,” Proc. Natl. Acad. Sci. U.S.A. 101(24):8882-8887, Anderson et al. (2004) “An expanded genetic code with a functional quadruplet codon” Proc. Natl. Acad. Sci. U.S.A. 101(20):7566-7571, Ikeda et al. (2003) “Synthesis of a novel histidine analogue and its efficient incorporation into a protein in vivo,” Protein Eng. Des. Sel. 16(9):699-706, Chin et al. (2003) “An Expanded Eukaryotic Genetic Code,” Science 301(5635):964-967, James et al. (2001) “Kinetic characterization of ribonuclease S mutants containing photoisomerizable phenylazophenylalanine residues,” Protein Eng. Des. Sel. 14(12):983-991, Kohrer et al. (2001) “Import of amber and ochre suppressor tRNAs into mammalian cells: A general approach to site-specific insertion of amino acid analogues into proteins,” Proc. Natl. Acad. Sci. U.S.A. 98(25):14310-14315, Bacher et al. (2001) “Selection and Characterization of Escherichia coli Variants Capable of Growth on an Otherwise Toxic Tryptophan Analogue,” J. Bacteriol. 183(18):5414-5425, Hamano-Takaku et al. (2000) “A Mutant Escherichia coli Tyrosyl-tRNA Synthetase Utilizes the Unnatural Amino Acid Azatyrosine More Efficiently than Tyrosine,” J. Biol. Chem. 275(51):40324-40328, and Budisa et al. (2001) “Proteins with {beta}-(thienopyrrolyl)alanines as alternative chromophores and pharmaceutically active amino acids,” Protein Sci. 10(7):1281-1292. Amino acids can be merged into peptides, polypeptides, or proteins.

In the context of the present invention, the term “peptide” refers to a short polymer of amino acids linked by peptide bonds. It has the same chemical (peptide) bonds as proteins, but is commonly shorter in length. The shortest peptide is a dipeptide, consisting of two amino acids joined by a single peptide bond. There can also be a tripeptide, tetrapeptide, pentapeptide, etc. Typically, a peptide has a length of up to 4, 6, 8, 10, 12, 15, 18 or 20 amino acids. A peptide has an amino end and a carboxyl end, unless it is a cyclic peptide.

In the context of the present invention, the term “polypeptide” refers to a single linear chain of amino acids bonded together by peptide bonds and typically comprises at least about 21 amino acids, i.e. at least 21, 22, 23, 24, 25, etc. amino acids. A polypeptide can be one chain of a protein that is composed of more than one chain or it can be the protein itself if the protein is composed of one chain.

In the context of the different aspects of present invention, the term “protein” refers to a molecule comprising one or more polypeptides that resume a secondary and tertiary structure and additionally refers to a protein that is made up of several polypeptides, i.e. several subunits, forming quaternary structures. The protein has sometimes non-peptide groups attached, which can be called prosthetic groups or cofactors.

In particular, the term “peptide variant”, “polypeptide variant”, “protein variant” is to be understood as a peptide, polypeptide, or protein which differs in comparison to the peptide, polypeptide, or protein from which it is derived by one or more changes in the amino acid sequence, as for example for mutant strains (variants). The peptide, polypeptide, or protein, from which a peptide, polypeptide, or protein variant is derived, is also known as the parent peptide, polypeptide, or protein. Further, the variants usable in the present invention may also be derived from homologs, orthologs, or paralogs of the parent peptide, polypeptide, or protein or from artificially constructed variant, provided that the variant exhibits at least one biological activity of the parent peptide, polypeptide, or protein. The changes in the amino acid sequence may be amino acid exchanges, insertions, deletions, N-terminal truncations, or C-terminal truncations, or any combination of these changes, which may occur at one or several sites. A peptide, polypeptide, or protein variant may exhibit a total number of up to 200 (up to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200) changes in the amino acid sequence (i.e. exchanges, insertions, deletions, N-terminal truncations, and/or C-terminal truncations). The amino acid exchanges may be conservative and/or non-conservative. Alternatively or additionally, a “variant” as used herein, can be characterized by a certain degree of sequence identity to the parent peptide, polypeptide, or protein from which it is derived. More precisely, a peptide, polypeptide, or protein variant in the context of the present invention exhibits at least 80% sequence identity to its parent peptide, polypeptide, or protein. The sequence identity of peptide, polypeptide, or protein variants is over a continuous stretch of 20, 30, 40, 45, 50, 60, 70, 80, 90, 100 or more amino acids.

The term “substitution”, in accordance with the present invention, refers to the replacement of an amino acid with another amino acid. Thus, the total number of amino acids remains the same. The deletion of an amino acid at a certain position or the introduction of one (or more) amino acid(s) at a different position, respectively, is explicitly not encompassed by the term “substitution”.

The term “conservative amino acid substitution” refers to a substitution in which an amino acid residue is substituted by another amino acid residue having a side chain (R group) with similar chemical properties (e.g., charge or hydrophobicity). In general, a conservative amino acid substitution will not substantially change the functional properties of a protein. Such similarities include e.g. a similarity in polarity, charge, solubility, hydrophobicity, hydrophilicity, and/or the amphipathic nature of the residues involved. In one embodiment a conservative amino acid substitution is a substitution of one amino acid for another one as comprised within one of the following groups, (i) nonpolar (hydrophobic) amino acids including alanine, valine, leucine, isoleucine, proline, phenylalanine, tyrosine, tryptophan, and methionine; (ii) polar neutral amino acids including glycine, serine, threonine, cysteine, asparagine, and glutamine; (iii) positively charged (basic) amino acids including arginine, lysine, and histidine; and (iv) negatively charged (acidic) amino acids including aspartic acid and glutamic acid.

The term “specific binding agent” refers to a natural or non-natural molecule that specifically binds to a target. Examples of specific binding agents include, but are not limited to, proteins, peptides and nucleic acids.

The term “antigen (Ag)” is a molecule or molecular structure, which is bound to by an antigen-specific antibody (Ab) or B cell antigen receptor (BCR). The presence of an antigen in the body normally triggers an immune response. In the body, each antibody is specifically produced to match an antigen after cells of the immune system come into contact with it; this allows a precise identification or matching of the antigen and the initiation of a tailored response. In most cases, an antibody can only react to and bind one specific antigen; in some instances, however, antibodies may cross-react and bind more than one antigen. Antigens are normally proteins, peptides (amino acid chains) and polysaccharides (chains of monosaccharides/simple sugars) or combinations thereof.

The term “binding preference” or “binding preference” indicates that under otherwise comparable conditions one out of two alternative antigens or targets is better bound than the other one.

Typically, the term “antibody” as used herein refers to secreted immunoglobulins which lack the transmembrane region and can thus, be released into the bloodstream and body cavities. The type of heavy chain present defines the class of antibody, i.e. these chains are found in IgA, IgD, IgE, IgG, and IgM antibodies, respectively, each performing different roles, and directing the appropriate immune response against different types of antigens. Distinct heavy chains differ in size and composition; and may comprise approximately 450 amino acids (Janeway et al. (2001) Immunobiology, Garland Science). IgA is found in mucosal areas, such as the gut, respiratory tract and urogenital tract, as well as in saliva, tears, and breast milk and prevents colonization by pathogens (Underdown & Schiff (1986) Annu. Rev. Immunol. 4:389-417). IgD mainly functions as an antigen receptor on B cells that have not been exposed to antigens and is involved in activating basophils and mast cells to produce antimicrobial factors (Geisberger et al. (2006) Immunology 118:429-437; Chen et al. (2009) Nat. Immunol. 10:889-898). IgE is involved in allergic reactions via its binding to allergens triggering the release of histamine from mast cells and basophils. IgE is also involved in protecting against parasitic worms (Pier et al. (2004) Immunology, Infection, and Immunity, ASM Press). IgG provides the majority of antibody-based immunity against invading pathogens and is the only antibody isotype capable of crossing the placenta to give passive immunity to fetus (Pier et al. (2004) Immunology, Infection, and Immunity, ASM Press). In humans there are four different IgG subclasses (IgG1, 2, 3, and 4), named in order of their abundance in serum with IgGl being the most abundant (˜66%), followed by IgG2 (˜23%), IgG3 (˜7%) and IgG (˜4%). The biological profile of the different IgG classes is determined by the structure of the respective hinge region. IgM is expressed on the surface of B cells in a monomeric form and in a secreted pentameric form with very high avidity. IgM is involved in eliminating pathogens in the early stages of B cell mediated (humoral) immunity before sufficient IgG is produced (Geisberger et al. (2006) Immunology 118:429-437). Antibodies are not only found as monomers but are also known to form dimers of two Ig units (e.g. IgA), tetramers of four Ig units (e.g. IgM of teleost fish), or pentamers of five Ig units (e.g. mammalian IgM). Antibodies are typically made of four polypeptide chains comprising two identical heavy chains and identical two light chains which are connected via disulfide bonds and resemble a “Y”-shaped macro-molecule. Each of the chains comprises a number of immunoglobulin domains out of which some are constant domains and others are variable domains. Immunoglobulin domains consist of a 2-layer sandwich of between 7 and 9 antiparallel˜—strands arranged in two˜—sheets. Typically, the heavy chain of an antibody comprises four Ig domains with three of them being constant (CH domains: CHI. CH2. CH3) domains and one of the being a variable domain (V H). The light chain typically comprises one constant Ig domain (CL) and one variable Ig domain (V L). Exemplified, the human IgG heavy chain is composed of four Ig domains linked from N- to C-terminus in the order VwCH1-CH2-CH3 (also referred to as VwCy1-Cy2-Cy3), whereas the human IgG light chain is composed of two immunoglobulin domains linked from N- to C-terminus in the order VL-CL, being either of the kappa or lambda type (VK-CK or VA.-CA). Exemplified, the constant chain of human IgG comprises 447 amino acids. Throughout the present specification and claims, the numbering of the amino acid positions in an immunoglobulin are that of the “EU index” as in Kabat, E. A., Wu, T. T., Perry, H. M., Gottesman, K. S., and Foeller, C., (1991) Sequences of proteins of immunological interest, 5*ed. U.S. Department of Health and Human Service, National Institutes of Health, Bethesda, MD. The “EU index as in Kabat” refers to the residue numbering of the human IgG lEU antibody. Accordingly, CH domains in the context of IgG are as follows: “CHI” refers to amino acid positions 118-220 according to the EU index as in Kabat; “CH2” refers to amino acid positions 237-340 according to the EU index as in Kabat; and “CH3” refers to amino acid positions 341-44 7 according to the EU index as in Kabat.

The terms “full-length antibody” “intact antibody” and “whole antibody” are used herein interchangeably to refer to an antibody in its substantially intact form, not antibody fragments as defined below. The terms particularly refer to an antibody with heavy chains that contain an Fc region.

Papain digestion of antibodies produces two identical antigen binding fragments, called “Fab fragments” (also referred to as “Fab portion” or “Fab region”) each with a single antigen binding site, and a residual “Fe fragment” (also referred to as “Fe portion” or “Fe region”) whose name reflects its ability to crystallize readily. The crystal structure of the human IgG Fe region has been determined (Deisenhofer (1981) Biochemistry 20:2361-2370). In IgG, IgA and IgD isotypes, the Fe region is composed of two identical protein fragments, derived from the CH2 and CH3 domains of the antibody's two heavy chains; in IgM and IgE isotypes, the Fe regions contain three heavy chain constant domains (CH2-4) in each polypeptide chain. In addition, smaller immunoglobulin molecules exist naturally or have been constructed artificially. The term “Fab′ fragment” refers to a Fab fragment additionally comprise the hinge region of an Ig molecule whilst “F(ab′)2 fragments” are understood to comprise two Fab′ fragments being either chemically linked or connected via a disulfide bond. Whilst “single domain antibodies (sdAb)” (Desmyter et al. (1996) Nat. Structure Biol. 3:803-811) and “Nanobodies” only comprise a single VH domain, “single chain Fv (scFv)” fragments comprise the heavy chain variable domain joined via a short linker peptide to the light chain variable domain (Huston et al. (1988) Proc. Natl. Acad. Sci. USA 85, 5879-5883). Divalent single-chain variable fragments (di-scFvs) can be engineered by linking two scFvs (scFvA-scFvB). This can be done by producing a single peptide chain with two VH and two VL regions, yielding “tandem scFvs” (VHA-VLA-VHB-VLB). Another possibility is the creation of scFvs with linkers that are too short for the two variable regions to fold together, forcing scFvs to dimerize. Usually linkers with a length of 5 residues are used to generate these dimers. This type is known as “diabodies”. Still shorter linkers (one or two amino acids) between a V H and V L domain lead to the formation of monospecific trimers, so-called “triabodies” or “tribadies”. Bispecific diabodies are formed by expressing to chains with the arrangement VHA-VLB and VHB-VLA or VLA-VHB and VLB-VHA, respectively. Singlechain diabodies (scDb) comprise a VHA-VLB and a VHB-VLA fragment which are linked by a linker peptide (P) of 12-20 amino acids, preferably 14 amino acids, (VHA-VLB-P-VHB-VLA). “Bispecific T-cell engagers (BiTEs)” are fusion proteins consisting of two scFvs of different antibodies wherein one of the scFvs binds to T cells via the CD3 receptor, and the other to a tumor cell via a tumor specific molecule (Kufer et al. (2004) Trends Biotechnol. 22:238-244). Dual affinity retargeting molecules (“DART” molecules) are diabodies additionally stabilized through a C-terminal disulfide bridge.

Accordingly, the term “antibody fragments” refers to a portion of an intact antibody, preferably comprising the antigen-binding region thereof. Antibody fragments include but are not limited to Fab, Fab′, F(ab′)2, Fv fragments; diabodies; sdAb, nanobodies, scFv, di-scFvs, tandem scFvs, triabodies, diabodies, scDb, BiTEs, and DARTs.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, MD (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any vertebrate species can be assigned to one of two clearly distinct types, called kappa (κ) and lambda (λ), based on the amino acid sequences of their constant domains.

A “naked antibody” for the purposes herein is an antibody that is not conjugated to any additionally moiety, such as e.g. a cytotoxic moiety or a label (e.g. a radiolabel).

The term “hypervariable region,” “HVR,” or “HV,” when used herein refers to the regions of an antibody-variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al. Immunity 13:37-45 (2000); Johnson and Wu in Methods in Molecular Biology 248:1-25 (Lo, ed., Human Press, Totowa, NJ, 2003). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996). A number of HVR delineations are in use and are encompassed herein. The HVRs that are Kabat complementarity-determining regions (CDRs) are based on sequence variability and are the most commonly used (Kabat et al., Sequences of Proteins of Immunological Interest, 5th Ed. Public Health Service, National Institutes of Health, Bethesda, MD (1991)). Chothia refers instead to the location of the structural loops (Chothia and Lesk J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact | |
|---|---|---|---|---|---|
| L1 | L24-L34 | L24-L34 | L26-L32 | L30-L36 | |
| L2 | L50-L56 | L50-L56 | L50-L52 | L46-L55 | |
| L3 | L89-L97 | L89-L97 | L91-L96 | L89-L96 | |
| H1 | H31-H35B | H26-H35B | H26-H32 | H30-H35B | (Kabat Numbering) |
| H1 | H31-H35 | H26-H35 | H26-H32 | H30-H35 | (Chothia Numbering) |
| H2 | H50-H65 | H50-H58 | H53-H55 | H47-H58 | |
| H3 | H95-H102 | H95-H102 | H96-H101 | H93-H101 | |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

A light chain variable domain/sequence consists of framework regions (FRs) and complementarity-determining regions (CDRs) as represented in formula I:

FR-L1-CDR-L1-FR-L2-CDR-L2-FR-L3-CDR-L3-FR-L4

A heavy chain variable domain/sequence consists of FRs and CDRs as represented in formula II:

FR-H1-CDR-H1-FR-H2-CDR-H2-FR-H3-CDR-H3-FR-H4

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra Using this numbering system, the actual linear amino acid sequence may contain fewer or additional amino acids corresponding to a shortening of, or insertion into, a FR or CDR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The “EU index as in Kabat” refers to the residue numbering of the human IgG lEU antibody. Accordingly, CH domains in the context of IgG are as follows: “CHI” refers to amino acid positions 118-220 according to the EU index as in Kabat; “CH2” refers to amino acid positions 237-340 according to the EU index as in Kabat; and “CH3” refers to amino acid positions 341-44 7 according to the EU index as in Kabat.

The term “binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the equilibrium dissociation constant (K_{D}). This constant is also the ratio of the “on-rate” or “association rate constant” (kₐ) and “off-rate” or “dissociation rate constant” (k_{d}). Two antibodies may have the same affinity, but one may have both a high on- and off-rate constant, while the other may have both a low on- and off-rate constant. Whilst the association rate constant kₐ [M⁻¹ s⁻¹] defines the complex formation velocity for the antibody/antigen-complex, the dissociation rate constant [s⁻¹] defines the antibody/antigen complex stability as the decay per second. Recalculated according to the formula t/2 diss=ln(2)/(kd*60), the antibody/antigen complex half-life in minutes, represents a descriptive parameter.

Affinity can be measured by common methods known in the art, including but not limited to surface plasmon resonance based assay (such as the BTAcore assay as described in PCT Application Publication No. WO2005/012359); enzyme-linked immunoabsorbent assay (ELISA); and competition assays (e.g. RIA's). Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity are described in the following.

The kₐ and k_{d}-values may be measured using methods well-known in the art, e.g by using surface-plasmon resonance assays using a BIACORE*-2000 or a BIACORE®-3000 instrument (BIAcore, Inc., Piscataway, NJ) at 25° C. with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve a sufficient high density of coupled protein. Following the injection of antigen, 1 M ethanolamine is injected to block unreacted groups. For kinetics measurements, two-fold serial dilutions of Fab (0.78 nM to 500 nM) are injected in PBS with 0.05% TWEEN 20™ surfactant (PBST) at 25° C. at a flow rate of approximately 25 μl/min. Association rates (kₐ) and dissociation rates (k_{d}) are calculated using a simple one-to-one Langmuir binding model (BIAcore® Evaluation Software version 3.2) by simultaneously fitting the association and dissociation sensorgrams. The equilibrium dissociation constant (K_{D}) is calculated as the ratio k_{d}/kₐ. See, e.g., Chen et al., J. Mol. Biol. 293:865-881 (1999). If the on-rate exceeds 10⁶ M⁻¹s⁻¹ by the surface-plasmon resonance assay above, then the on-rate can be determined by using a fluorescent quenching technique that measures the increase or decrease in fluorescence-emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow-equipped spectrophotometer (Aviv Instruments) or a 8000-series SLM-AMINCO™ spectrophotometer (ThermoSpectronic) with a stirred cuvette.

The term “monoclonal antibody” (mAb)” as used herein refers to monospecific antibodies that are made by identical immune cells which are clones of a unique parent cell and are thus, all reactive to the identical epitope of a given target molecule. In contrast “polyclonal antibodies” are made from several different immune cells and thus, target different epitopes of a given target molecule. Accordingly, monoclonal antibodies have monovalent affinity, i.e. they bind to the same epitope, whereas polyclonal antibodies bind to several different epitopes of the same target. In contrast to polyclonal antibody preparations, which typically include different antibodies directed against different determinants (epitopes), each monoclonal antibody of a monoclonal-antibody preparation is directed against a single determinant on an antigen. In addition to their specificity, monoclonal-antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the Monoclonal antibodies to be used in accordance with the present invention may be made by a variety of techniques, including, for example, but not limited to the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, PNAS USA 101(34): 12467-12472 (2004); and Lee et al., J. Immunol. Methods 284(1-2): 119-132(2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., PNAS USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

Antibody may further comprise an “effector group” such as e.g. a “tag” or a “label”. The term “tag” refers to those effector groups which provide the antibody with the ability to bind to or to be bound to other molecules. Examples of tags include but are not limited to e.g. His tags which are attached to the antigen sequence to allow for its purification. Tag may also include a partner of a bioaffine binding pair which allows the antigen to be bound by the second partner of the binding pair. The term “bioaffine binding pair” refers to two partner molecules (i.e. two partners in one pair) having a strong affinity to bind to each other. Examples of partners of bioaffine binding pairs are a) biotin or biotin analogs/avidin or streptavidin; b) Haptens/anti-hapten antibodies or antibody fragments (e.g. digoxin/anti-digoxin antibodies); c) Saccharides/lectins; d) complementary oligonucleotide sequences (e.g. complementary LNA sequences), and in general e) ligands/receptors.

The term “label” refers to those effector groups which allow for the detection of the antigen. Label include but are not limited to spectroscopic, photochemical, biochemical, immunochemical, or chemical, label. Exemplified, suitable labels include fluorescent dyes, luminescent or electrochemiluminescent complexes (e.g. ruthenium or iridium complexes), electron-dense reagents, and enzymatic label.

“Sandwich immunoassays” are broadly used in the detection of an analyte of interest. In such assay the analyte is “sandwiched” in between a first antibody and a second antibody. Typically, a sandwich assay requires that capture and detection antibody bind to different, non-overlapping epitopes on an analyte of interest. By appropriate means such sandwich complex is measured and the analyte thereby quantified. In a typical sandwich-type assay, a first antibody bound to the solid phase or capable of binding thereto and a detectably-labeled second antibody each bind to the analyte at different and non-overlapping epitopes. The first analyte-specific binding agent (e.g. an antibody) is either covalently or passively bound to a solid surface. The solid surface is typically glass or a polymer, the most commonly used polymers being cellulose, polyacrylamide, nylon, polystyrene, polyvinyl chloride, or polypropylene. The solid supports may be in the form of particles, tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g., from room temperature to 40° C. such as between 25° C. and 37° C. inclusive) to allow for binding between the first or capture antibody and the corresponding antigen. Following the incubation period, the solid phase, comprising the first or capture antibody and bound thereto the antigen can be washed, and incubated with a secondary or labeled antibody binding to another epitope on the antigen. The second antibody is linked to a reporter molecule which is used to indicate the binding of the second antibody to the complex of first antibody and the antigen of interest.

An extremely versatile alternative sandwich assay format includes the use of a solid phase coated with the first partner of a binding pair, e.g. paramagnetic streptavidin-coated microparticles. Such microparticles are mixed and incubated with an analyte-specific binding agent bound to the second partner of the binding pair (e.g. a biotinylated antibody), a sample suspected of comprising or comprising the analyte, wherein said second partner of the binding pair is bound to said analyte-specific binding agent, and a second analyte-specific binding agent which is detectably labeled. As obvious to the skilled person these components are incubated under appropriate conditions and for a period of time sufficient for binding the labeled antibody via the analyte, the analyte-specific binding agent (bound to) the second partner of the binding pair and the first partner of the binding pair to the solid phase microparticles. As appropriate such assay may include one or more washing step(s). The term “detectably labeled” encompasses labels that can be directly or indirectly detected. Directly detectable labels either provide a detectable signal or they interact with a second label to modify the detectable signal provided by the first or second label, e.g. to give FRET (fluorescence resonance energy transfer). Labels such as fluorescent dyes and luminescent (including chemiluminescent and electrochemiluminescent) dyes (Briggs et al “Synthesis of Functionalised Fluorescent Dyes and Their Coupling to Amines and Amino Acids,” J. Chem. Soc., Perkin-Trans. 1 (1997) 1051-1058) provide a detectable signal and are generally applicable for labeling. In one embodiment detectably labeled refers to a label providing or inducible to provide a detectable signal, i.e. to a fluorescent label, to a luminescent label (e.g. a chemiluminescent label or an electrochemiluminescent label), a radioactive label or a metal-chelate based label, respectively.

Numerous labels (also referred to as dyes) are available which can be generally grouped into the following categories, all of them together and each of them representing embodiments according the present disclosure:

### (a) Fluorescent Dyes

Fluorescent dyes are e.g. described by Briggs et al “Synthesis of Functionalized Fluorescent Dyes and Their Coupling to Amines and Amino Acids,” J. Chem. Soc., Perkin-Trans. 1 (1997) 1051-1058).

Fluorescent labels or fluorophores include rare earth chelates (europium chelates), fluorescein type labels including FITC, 5-carboxyfluorescein, 6-carboxy fluorescein; rhodamine type labels including TAMRA; dansyl; Lissamine; cyanines; phycoerythrins; Texas Red; and analogs thereof. The fluorescent labels can be conjugated to an aldehyde group comprised in target molecule using the techniques disclosed herein. Fluorescent dyes and fluorescent label reagents include those which are commercially available from Invitrogen/Molecular Probes (Eugene, Oregon, USA) and Pierce Biotechnology, Inc. (Rockford, Ill.).

### (b) Luminescent Dyes

Luminescent dyes or labels can be further subcategorized into chemiluminescent and electrochemiluminescent dyes.

The different classes of chemiluminogenic labels include luminol, acridinium compounds, coelenterazine and analogues, dioxetanes, systems based on peroxyoxalic acid and their derivatives. For immunodiagnostic procedures predominantly acridinium based labels are used (a detailed overview is given in Dodeigne C. et al., Talanta 51 (2000) 415-439).

The labels of major relevance used as electrochemiluminescent labels are the Ruthenium- and the Iridium-based electrochemiluminescent complexes, respectively. Electrochemiluminescense (ECL) proved to be very useful in analytical applications as a highly sensitive and selective method. It combines analytical advantages of chemiluminescent analysis (absence of background optical signal) with ease of reaction control by applying electrode potential. In general Ruthenium complexes, especially [Ru (Bpy)3]2+(which releases a photon at ˜620 nm) regenerating with TPA (Tripropylamine) in liquid phase or liquid-solid interface are used as ECL-labels.

Electrochemiluminescent (ECL) assays provide a sensitive and precise measurement of the presence and concentration of an analyte of interest. Such techniques use labels or other reactants that can be induced to luminesce when electrochemically oxidized or reduced in an appropriate chemical environment. Such electrochemiluminescense is triggered by a voltage imposed on a working electrode at a particular time and in a particular manner. The light produced by the label is measured and indicates the presence or quantity of the analyte. For a fuller description of such ECL techniques, reference is made to U.S. Pat. Nos. 5,221,605, 5,591,581, 5,597,910, PCT published application WO90/05296, PCT published application WO92/14139, PCT published application WO90/05301, PCT published application WO96/24690, PCT published application US95/03190, PCT application US97/16942, PCT published application US96/06763, PCT published application WO95/08644, PCT published application WO96/06946, PCT published application WO96/33411, PCT published application WO87/06706, PCT published application WO96/39534, PCT published application WO96/41175, PCT published application WO96/40978, PCT/US97/03653 and U.S. patent application Ser. No. 08/437,348 (U.S. Pat. No. 5,679,519). Reference is also made to a 1994 review of the analytical applications of ECL by Knight, et al. (Analyst, 1994, 119: 879-890) and the references cited therein. In one embodiment the method according to the present description is practiced using an electrochemiluminescent label.

Recently also Iridium-based ECL-labels have been described (WO2012107419).
(c) Radioactive labels make use of radioisotopes (radionuclides), such as 3H, 11C, 14C, 18F, 32P, 35S, 64Cu, 68Gn, 86Y, 89Zr, 99TC, 111In, 123I, 124I, 125I, 131I, 133Xe, 177Lu, 211At, or 131Bi.
(d) Metal-chelate complexes suitable as labels for imaging and therapeutic purposes are well-known in the art (US 2010/0111861; U.S. Pat. Nos. 5,342,606; 5,428,155; 5,316,757; 5,480,990; 5,462,725; 5,428,139; 5,385,893; 5,739,294; 5,750,660; 5,834,461; Hnatowich et al, J. Immunol. Methods 65 (1983) 147-157; Meares et al, Anal. Biochem. 142 (1984) 68-78; Mirzadeh et al, Bioconjugate Chem. 1 (1990) 59-65; Meares et al, J. Cancer (1990), Suppl. 10:21-26; Izard et al, Bioconjugate Chem. 3 (1992) 346-350; Nikula et al, Nucl. Med. Biol. 22 (1995) 387-90; Camera et al, Nucl. Med. Biol. 20 (1993) 955-62; Kukis et al, J. Nucl. Med. 39 (1998) 2105-2110; Verel et al., J. Nucl. Med. 44 (2003) 1663-1670; Camera et al, J. Nucl. Med. 21 (1994) 640-646; Ruegg et al, Cancer Res. 50 (1990) 4221-4226; Verel et al, J. Nucl. Med. 44 (2003) 1663-1670; Lee et al, Cancer Res. 61 (2001) 4474-4482; Mitchell, et al, J. Nucl. Med. 44 (2003) 1105-1112; Kobayashi et al Bioconjugate Chem. 10 (1999) 103-111; Miederer et al, J. Nucl. Med. 45 (2004) 129-137; DeNardo et al, Clinical Cancer Research 4 (1998) 2483-90; Blend et al, Cancer Biotherapy & Radiopharmaceuticals 18 (2003) 355-363; Nikula et al J. Nucl. Med. 40 (1999) 166-76; Kobayashi et al, J. Nucl. Med. 39 (1998) 829-36; Mardirossian et al, Nucl. Med. Biol. 20 (1993) 65-74; Roselli et al, Cancer Biotherapy & Radiopharmaceuticals, 14 (1999) 209-20).

A “particle” as used herein means a small, localized object to which can be ascribed a physical property such as volume, mass or average size. Particles may accordingly be of a symmetrical, globular, essentially globular or spherical shape, or be of an irregular, asymmetric shape or form. The size of a particle may vary. The term “microparticle” refers to particles with a diameter in the nanometer and micrometer range.

Microparticles as defined herein above may comprise or consist of any suitable material known to the person skilled in the art, e.g. they may comprise or consist of or essentially consist of inorganic or organic material. Typically, they may comprise or consist of or essentially consist of metal or an alloy of metals, or an organic material, or comprise or consist of or essentially consist of carbohydrate elements. Examples of envisaged material for microparticles include agarose, polystyrene, latex, polyvinyl alcohol, silica and ferromagnetic metals, alloys or composition materials. In one embodiment the microparticles are magnetic or ferromagnetic metals, alloys or compositions. In further embodiments, the material may have specific properties and e.g. be hydrophobic, or hydrophilic. Such microparticles typically are dispersed in aqueous solutions and retain a small negative surface charge keeping the microparticles separated and avoiding non-specific clustering.

In one embodiment of the present invention, the microparticles are paramagnetic microparticles and the separation of such particles in the measurement method according to the present disclosure is facilitated by magnetic forces. Magnetic forces are applied to pull the paramagnetic or magnetic particles out of the solution/suspension and to retain them as desired while liquid of the solution/suspension can be removed and the particles can e.g. be washed.

A “kit” is any manufacture (e.g. a package or container) comprising at least one reagent, e.g., a medicament for treatment of a disorder, or a probe for specifically detecting a biomarker gene or protein of the invention. The kit is preferably promoted, distributed, or sold as a unit for performing the methods of the present invention. Typically, a kit may further comprise carrier means being compartmentalized to receive in close confinement one or more container means such as vials, tubes, and the like In particular, each of the container means comprises one of the separate elements to be used in the method of the first aspect. Kits may further comprise one or more other containers comprising further materials including but not limited to buffers, diluents, filters, needles, syringes, and package inserts with instructions for use. A label may be present on the container to indicate that the composition is used for a specific application, and may also indicate directions for either in vivo or in vitro use. The computer program code may be provided on a data storage medium or device such as a optical storage medium (e.g., a Compact Disc) or directly on a computer or data processing device. Moreover, the kit may, comprise standard amounts for the biomarkers as described elsewhere herein for calibration purposes.

A “package insert” is used to refer to instructions customarily included in commercial packages of therapeutic products or medicaments, that contain information about the indications, usage, dosage, administration, contraindications, other therapeutic products to be combined with the packaged product, and/or warnings concerning the use of such therapeutic products or medicaments, etc.

### Embodiments

Currently available PCR format diagnostic assays for detecting SARS CoV-2 virus in patient's samples require several hours for the results to be available. They are thus not sufficient to fulfill the high demand for Coronavirus tests in the currently ongoing pandemic. Rapid point of care antigen test provide much faster results, but often do not exhibit the required sensitivity and/or specificity as required for a reliable diagnosis. To provide for the high demand of reliable diagnostic results in the pandemic, we developed a high-throughput antigen assay using highly-specific antibodies.

In a first aspect, the present invention relates to an (isolated) monoclonal antibody or antigen-binding fragment thereof that binds to the RBD of the spike protein of SARS-CoV-2 virus
a) with an association rate constant (kₐ) of more than 2.5E+06 M⁻¹s⁻¹, as determined by surface plasmon resonance,
and/or
b) with a dissociation rate constant (k_{d}) of less than 5.0E-03 s⁻¹, as determined by surface plasmon resonance,
and/or
c) with a half-life time of t/2diss of 4 minutes or more, as determined by surface plasmon resonance,
and/or
d) with a 1:1 or 1:2 stoichiometry.

In particular embodiments, the antibody of the first aspect is a neutralizing antibody.

In particular embodiments, the antibody of the first aspect binds to the RBD of the spike protein of SARS-CoV-2 virus wildtype and mutant strains (variants).

In particular embodiments, the antibody has an association rate constant (kₐ) of more than 2.0E+06 M⁻¹s⁻¹, in particular of more than 2.5E+06 M⁻¹s. In particular embodiments, the antibody has an association rate constant (kₐ) of more than 2.7E+06 M⁻¹s⁻¹, in particular of more than 3.0E+06 M⁻¹s. In particular embodiments, the antibody has an association rate constant (kₐ) of more than 3.3E+06 M⁻¹s⁻¹.

In particular embodiments, the antibody has a dissociation rate constant (k_{d}) of less than 5.0E-03 s⁻¹, in particular less than 4.5E-03 s⁻¹, in particular less than 4.0E-03 s⁻¹, in particular 3.0E-03 s⁻¹, in particular of less than 2.7E-03 s⁻¹. In particular embodiments, the antibody has a dissociation rate constant (k_{d}) of less than 2.6E-03 s⁻¹, in particular of less than 1.1E-03 s⁻¹.

In particular embodiments, the antibody has an antibody/antigen complex half-life time of t/2diss of 4 minutes or more, t/2diss of 6 minutes or more, in particular of t/2diss of 11 minutes or more.

In particular embodiments, the antibody has an association rate constant (kₐ) of 3.3E+06 M⁻¹s⁻¹ and a dissociation rate constant (k_{d}) of 1.1E-03 s⁻¹. In particular embodiments, the antibody has an antibody/antigen complex half-life time of t/2diss of 11 min.

In particular embodiments, the antibody has an association rate constant (kₐ) of 2.7E+06 M⁻¹s⁻¹ and a dissociation rate constant (k_{d}) of 2.7E-03 s⁻¹. In particular embodiments, the antibody has an antibody/antigen complex half-life time of t/2diss of 4 min.

In particular embodiments, the antibody has an association rate constant (kₐ) of 3.0E+06 M⁻¹s⁻¹ and a dissociation rate constant (k_{d}) of 2.6E-03 s⁻¹. In particular embodiments, the antibody has an antibody/antigen complex half-life time of t/2diss of 4 min.

In particular embodiments, the antibody has an association rate constant (kₐ) of 2.5E+06 M⁻¹s⁻¹ and a dissociation rate constant (k_{d}) of 1.9E-03 s⁻¹. In particular embodiments, the antibody has an antibody/antigen complex half-life time of t/2diss of 6 min.

In particular embodiments, the antibody has a sequence as described for any of aspects 2 to 5 below.

In embodiments, the antibody or antigen-binding fragment of the present invention is an isolated antibody or antigen-binding fragment. Thus, the antibody or antigen-binding fragment is an antibody or antigen-binding fragment, which has been purified. Purification of an antibody can be achieved by methods well-known in the art such as Size Exclusion Chromatography (SEC). Accordingly, the antibody or antigen-binding fragment shall have been isolated from the cells in which the antibody was produced. In some embodiments, an isolated antibody or antigen-binding fragment is purified to greater than 70% by weight of antibody as determined by, for example, the Lowry method, and in some embodiments, to greater than 80%, 90%, 95%, 96%, 97%, 98% or 99% by weight. In one preferred embodiment the isolated antibody or antigen-binding fragment according to the present invention is purified to greater than 90% purity as determined by SDS-PAGE under reducing conditions using Coomassie blue staining for protein detection.

In embodiments, the antibody or antigen-binding fragment thereof is a naked antibody or naked antigen-binding fragment. In embodiments, the antibody or antigen-binding fragment thereof further comprises a tag or a label. In particular embodiments, the tag allows to bind the antibody or antigen-binding fragment thereof directly or indirectly to a solid phase. In particular embodiments, the tag is a partner of a bioaffine binding pair. In particular embodiments, the tag is selected from the group consisting of biotin, digoxin, hapten, or complementary oligonucleotide sequences (in particular complementary LNA sequences). In particular embodiments, the tag is biotin.

In particular embodiments, the label allows for the detection of the antibody or antigen-binding fragment thereof. In particular embodiments, the label is an electrochemiluminescent ruthenium or iridium complex. In particular embodiments, the electrochemiluminescent ruthenium complex is a negatively charged electrochemiluminescent ruthenium complex. In particular embodiments, the label is a negatively charged electrochemiluminescent ruthenium complex which is present in the antigen with a stoichiometry of 1:1 to 15:1. In particular embodiments the stoichiometry is 2:1, 2.5:1, 3:1, 5:1, 10:1, or 15:1.

In a second aspect, the present invention relates to an isolated antibody or an antigen-binding fragment thereof, which
a) comprises CDR-H1, CDR-H2, CDR-H3, CDR-L1, CDR-L2 and CDR-L3 according to SEQ ID NO: 1, 2, 3, 4, 5, and 6, respectively,
b) binds to the same epitope as an antibody comprising CDR-H1, CDR-H2, CDR-H3, CDR-L1, CDR-L2 and CDR-L3 according to SEQ ID NO: 1, 2, 3, 4, 5, and 6, respectively,
   or
c) competes for binding to the RBD of the spike protein of SARS-CoV-2 virus with an antibody comprising CDR-H1, CDR-H2, CDR-H3, CDR-L1, CDR-L2 and CDR-L3 according to SEQ ID NO: 1, 2, 3, 4, 5, and 6, respectively.

In particular embodiments, the antibody or antigen-binding fragment thereof comprises CDRs comprising the sequences specifically recited above, i.e. without any amino acid variation.

In particular embodiments, the antibody or antigen-binding fragment thereof comprises one or more CDRs with sequence variations of the sequences recited above. In particular embodiments, the sequence variation comprises 1 or 2, in particular 1, amino acid alteration. In particular embodiments the 1 or 2 amino acids alterations are independently of each other amino acid deletions, amino acid additions, or amino acid substitutions. In particular embodiments, the amino acid substitution is a conservative amino acid substitution.

In particular embodiments, the antibody or antigen-binding fragment of the second aspect further
a) comprises FR-H1, FR-H2, FR-H3, FR-H4, FR-L1, FR-L2, FR-L3 and FR-L4 according to SEQ ID NO: 7, 8, 9, 10, 11, 12, 13, and 14, respectively,
b) binds to the same epitope as an antibody comprising FR-H1, FR-H2, FR-H3, FR-H4, FR-L1, FR-L2, FR-L3 and FR-L4 according to SEQ ID NO: 7, 8, 9, 10, 11, 12, 13, and 14, respectively,
or
c) competes for binding to the RBD of the spike protein of SARS-CoV-2 virus with an antibody comprising FR-H1, FR-H2, FR-H3, FR-H4, FR-L1, FR-L2, FR-L3 and FR-L4 according to SEQ ID NO: 7, 8, 9, 10, 11, 12, 13, and 14, respectively.

In particular embodiments, the antibody or antigen-binding fragment thereof comprises FRs comprising the sequences specifically recited above, i.e. without any amino acid variation.

In particular embodiments, the antibody or antigen-binding fragment thereof comprises one or more FRs with sequence variations of the sequences recited above. In particular embodiments, the sequence variation comprises up to 5, in particular 1, 2, 3, 4, or 5 amino acid alteration. In particular embodiments the up to 5, in particular 1, 2, 3, 4, or 5, amino acids alterations are independently of each other amino acid deletions, amino acid additions, or amino acid substitutions. In particular embodiments, the amino acid substitution is a conservative amino acid substitution.

In particular embodiments, the antibody or antigen-binding fragment of the second aspect
a) comprises a heavy chain variable domain having an amino acid sequence according to SEQ ID NO: 15 and a light chain variable domain having an amino acid sequence according to SEQ ID NO: 16
b) binds to the same epitope as an antibody comprising a heavy chain variable domain having an amino acid sequence according to SEQ ID NO: 15 and a light chain variable domain having an amino acid sequence according to SEQ ID NO: 16
or
c) competes for binding to the RBD of the spike protein of SARS-CoV-2 virus with an antibody comprising a heavy chain variable domain having an amino acid sequence according to SEQ ID NO: 15 and a light chain variable domain having an amino acid sequence according to SEQ ID NO: 16.

In particular embodiments, the antibody or antigen-binding fragment thereof comprises heavy chain variable domain and light chain variable domain comprising the sequences specifically recited above, i.e. without any amino acid variation.

In particular embodiments, the antibody or antigen-binding fragment thereof comprises heavy chain variable domain and light chain variable domain with sequence variations of the sequences recited above. In particular embodiments, the variant sequence is at least 85% identical to the sequences specifically recited above.

In one further embodiment, the identity is at least 90%. In a further embodiment the identity is at least 95% in particular at least 98%.

In particular embodiments, the antibody or antigen-binding fragment thereof binds to the RBD of the spike protein of SARS-CoV-2 virus
a) with an association rate constant (kₐ) of more than 2.0E+06 M⁻¹s⁻¹, as determined by surface plasmon resonance,
and/or
b) with a dissociation rate constant (k_{d}) of less than 3.0E-03 s⁻¹, as determined by surface plasmon resonance,
and/or
c) with a half-life time of t/2diss of 4 minutes or more, as determined by surface plasmon resonance, and/or
d) with a 1:1 or 1:2 stoichometry.

In particular embodiments, the antibody has an association rate constant (kₐ) of more than 2.5E+06 M⁻¹s. In particular embodiments, the antibody has an association rate constant (kₐ) of more than 2.7E+06 M⁻¹s⁻¹, in particular of more than 3.0E+06 M⁻¹s⁻¹. In particular embodiments, the antibody has an association rate constant (kₐ) of more than 3.3E+06 M⁻¹s⁻¹.

In particular embodiments, the antibody has a dissociation rate constant (k_{d}) of less than 5.0E-03 s⁻¹, in particular of less than 4.5E-03 s⁻¹, in particular of less than 4.0E-03 s⁻¹, in particular of less than 3.5E-03 s⁻¹, 3.0E-03 s⁻¹, in particular of less than 2.7E-03 s⁻¹. In particular embodiments, the antibody has a dissociation rate constant (k_{d}) of less than 2.6E-03 s⁻¹, in particular of less than 1.1E-03 s⁻¹.

In particular embodiments, the antibody has an antibody/antigen complex half-life time of 4 minutes or more, t/2diss of 6 minutes or more, in particular of t/2diss of 11 minutes or more.

In particular embodiments, the antibody has an association rate constant (kₐ) of 3.3E+06 M⁻¹s⁻¹ and a dissociation rate constant (k_{d}) of 1.1E-03 s⁻¹. In particular embodiments, the antibody has an antibody/antigen complex half-life time of t/2diss of 11 min. In particular embodiments, the antibody has an association rate constant (kₐ) of 3.3E+06 M⁻¹s⁻¹ and a dissociation rate constant (k_{d}) of 1.1E-03 s⁻¹ and an antibody/antigen complex half-life time of t/2diss of 11 min.

In embodiments, the antibody or antigen-binding fragment of the present invention is an isolated antibody or antigen-binding fragment. Thus, the antibody or antigen-binding fragment is an antibody or antigen-binding fragment, which has been purified. Purification of an antibody can be achieved by methods well-known in the art such as Size Exclusion Chromatography (SEC). Accordingly, the antibody or antigen-binding fragment shall have been isolated from the cells in which the antibody was produced. In some embodiments, an isolated antibody or antigen-binding fragment is purified to greater than 70% by weight of antibody as determined by, for example, the Lowry method, and in some embodiments, to greater than 80%, 90%, 95%, 96%, 97%, 98% or 99% by weight. In one preferred embodiment the isolated antibody or antigen-binding fragment according to the present invention is purified to greater than 90% purity as determined by SDS-PAGE under reducing conditions using Coomassie blue staining for protein detection.

In embodiments, the antibody or antigen-binding fragment thereof is a naked antibody or naked antigen-binding fragment. In embodiments, the antibody or antigen-binding fragment thereof further comprises a tag or a label. In particular embodiments, the tag allows to bind the antibody or antigen-binding fragment thereof directly or indirectly to a solid phase. In particular embodiments, the tag is a partner of a bioaffine binding pair. In particular embodiments, the tag is selected from the group consisting of biotin, digoxin, hapten, or complementary oligonucleotide sequences (in particular complementary LNA sequences). In particular embodiments, the tag is biotin.

In particular embodiments, the label allows for the detection of the antibody or antigen-binding fragment thereof. In particular embodiments, the label is an electrochemiluminescent ruthenium or iridium complex. In particular embodiments, the electrochemiluminescent ruthenium complex is a negatively charged electrochemiluminescent ruthenium complex. In particular embodiments, the label is a negatively charged electrochemiluminescent ruthenium complex which is present in the antigen with a stoichiometry of 1:1 to 15:1. In particular embodiments the stoichiometry is 2:1, 2.5:1, 3:1, 5:1, 10:1, or 15:1.

In particular embodiments, the antibody of the second aspect binds to the RBD of the spike protein of SARS-CoV-2 virus wildtype and mutant strains (variants).

In a third aspect, the present invention relates to an antibody or an antigen-binding fragment thereof, which
a) comprises CDR-H1, CDR-H2, CDR-H3, CDR-L1, CDR-L2 and CDR-L3 according to SEQ ID NO: 17, 18, 19, 20, 21, and 22, respectively,
b) binds to the same epitope as an antibody comprising CDR-H1, CDR-H2, CDR-H3, CDR-L1, CDR-L2 and CDR-L3 according to SEQ ID NO: 17, 18, 19, 20, 21, and 22, respectively,
or
c) which competes for binding to the RBD of the spike protein of SARS-CoV-2 virus with an antibody comprising CDR-H1, CDR-H2, CDR-H3, CDR-L1, CDR-L2 and CDR-L3 according to SEQ ID NO: 17, 18, 19, 20, 21 and 22, respectively.

In particular embodiments, the antibody or antigen-binding fragment thereof comprises CDRs comprising the sequences specifically recited above, i.e. without any amino acid variation.

In particular embodiments, the antibody or antigen-binding fragment thereof comprises one or more CDRs with sequence variations of the sequences recited above. In particular embodiments, the sequence variation comprises 1 or 2, in particular 1, amino acid alteration. In particular embodiments the 1 or 2 amino acids alterations are independently of each other amino acid deletions, amino acid additions, or amino acid substitutions. In particular embodiments, the amino acid substitution is a conservative amino acid substitution.

In particular embodiments, the antibody or antigen-binding fragment of the third aspect further
a) comprises FR-H1, FR-H2, FR-H3, FR-H4, FR-L1, FR-L2, FR-L3, and FR-L4 according to SEQ ID NO: 23, 24, 25, 26, 27, 28, 29, and 30, respectively,
b) binds to the same epitope as an antibody comprising FR-H1, FR-H2, FR-H3, FR-H4, FR-L1, FR-L2, FR-L3, and FR-L4 according to SEQ ID NO: 23, 24, 25, 26, 27, 28, 29, and 30, respectively,
or
c) competes for binding to the RBD of the spike protein of SARS-CoV-2 virus with an antibody comprising FR-H1, FR-H2, FR-H3, FR-H4, FR-L1, FR-L2, FR-L3, and FR-L4 according to SEQ ID NO: 23, 24, 25, 26, 27, 28, 29, and 30, respectively.

In particular embodiments, the antibody or antigen-binding fragment thereof comprises FRs comprising the sequences specifically recited above, i.e. without any amino acid variation.

In particular embodiments, the antibody or antigen-binding fragment thereof comprises one or more FRs with sequence variations of the sequences recited above. In particular embodiments, the sequence variation comprises up to 5, in particular 1, 2, 3, 4, or 5 amino acid alteration. In particular embodiments the up to 5, in particular 1, 2, 3, 4, or 5, amino acids alterations are independently of each other amino acid deletions, amino acid additions, or amino acid substitutions. In particular embodiments, the amino acid substitution is a conservative amino acid substitution.

In particular embodiments, the antibody or antigen-binding fragment of the third aspect
a) comprises a heavy chain variable domain having an amino acid sequence according to SEQ ID NO: 31 and a light chain variable domain having an amino acid sequence according to SEQ ID NO: 32,
b) binds to the same epitope as an antibody comprising a heavy chain variable domain having an amino acid sequence according to SEQ ID NO: 31 and a light chain variable domain having an amino acid sequence according to SEQ ID NO: 32,
or
c) competes for binding to the RBD of the spike protein of SARS-CoV-2 virus with an antibody comprising a heavy chain variable domain having an amino acid sequence according to SEQ ID NO: 31 and a light chain variable domain having an amino acid sequence according to SEQ ID NO: 32.

In particular embodiments, the antibody or antigen-binding fragment thereof comprises heavy chain variable domain and light chain variable domain comprising the sequences specifically recited above, i.e. without any amino acid variation.

In particular embodiments, the antibody or antigen-binding fragment thereof comprises heavy chain variable domain and light chain variable domain with sequence variations of the sequences recited above. In particular embodiments, the variant sequence is at least 85% identical to the sequences specifically recited above.

In one further embodiment, the identity is at least 90%. In a further embodiment the identity is at least 95% in particular at least 98%.

In particular embodiments, the antibody or antigen-binding fragment thereof binds to the RBD of the spike protein of SARS-CoV-2 virus
a) with an association rate constant (kₐ) of more than 2.5E+06 M⁻¹s⁻¹, as determined by surface plasmon resonance,
and/or
b) with a dissociation rate constant (k_{d}) of less than 3.0E-03 s⁻¹, as determined by surface plasmon resonance,
and/or
c) with a half-life time of t/2diss of 4 minutes or more, as determined by surface plasmon resonance,
and/or
d) with a 1:1 or 1:2 stoichometry.

In particular embodiments, the antibody has an association rate constant (kₐ) of more than 2.5E+06 M⁻¹s. In particular embodiments, the antibody has an association rate constant (kₐ) of more than 2.7E+06 M⁻¹s⁻¹, in particular of more than 3.0E+06 M⁻¹s⁻¹. In particular embodiments, the antibody has an association rate constant (kₐ) of more than 3.3E+06 M⁻¹s⁻¹.

In particular embodiments, the antibody has a dissociation rate constant (k_{d}) of less than 5.0E-03 s⁻¹, in particular of less than 4.5E-03 s⁻¹, in particular of less than 4.0E-03 s⁻¹, in particular of less than 3.5E-03 s⁻¹, 3.0E-03 s⁻¹, in particular of less than 2.7E-03 s⁻¹. In particular embodiments, the antibody has a dissociation rate constant (k_{d}) of less than 2.6E-03 s⁻¹, in particular of less than 1.1E-03 s⁻¹.

In particular embodiments, the antibody has an antibody/antigen complex half-life time of 4 minutes or more, t/2diss of 6 minutes or more, in particular of t/2diss of 11 minutes or more.

In particular embodiments, the antibody has an association rate constant (kₐ) of 2.7E+06 M⁻¹s⁻¹ and a dissociation rate constant (k_{d}) of 2.7E-03 s⁻¹. In particular embodiments, the antibody has an antibody/antigen complex half-life time of t/2diss of 4 min. In particular embodiments, the antibody has an association rate constant (kₐ) of 2.7E+06 M⁻¹s⁻¹ and a dissociation rate constant (k_{d}) of 2.7E-03 s⁻¹ and an antibody/antigen complex half-life time of t/2diss of 4 min.

In embodiments, the antibody or antigen-binding fragment of the present invention is an isolated antibody or antigen-binding fragment. Thus, the antibody or antigen-binding fragment is an antibody or antigen-binding fragment, which has been purified. Purification of an antibody can be achieved by methods well-known in the art such as Size Exclusion Chromatography (SEC). Accordingly, the antibody or antigen-binding fragment shall have been isolated from the cells in which the antibody was produced. In some embodiments, an isolated antibody or antigen-binding fragment is purified to greater than 70% by weight of antibody as determined by, for example, the Lowry method, and in some embodiments, to greater than 80%, 90%, 95%, 96%, 97%, 98% or 99% by weight. In one preferred embodiment the isolated antibody or antigen-binding fragment according to the present invention is purified to greater than 90% purity as determined by SDS-PAGE under reducing conditions using Coomassie blue staining for protein detection.

In embodiments, the antibody or antigen-binding fragment thereof is a naked antibody or naked antigen-binding fragment. In embodiments, the antibody or antigen-binding fragment thereof further comprises a tag or a label. In particular embodiments, the tag allows to bind the antibody or antigen-binding fragment thereof directly or indirectly to a solid phase. In particular embodiments, the tag is a partner of a bioaffine binding pair. In particular embodiments, the tag is selected from the group consisting of biotin, digoxin, hapten, or complementary oligonucleotide sequences (in particular complementary LNA sequences). In particular embodiments, the tag is biotin.

In particular embodiments, the label allows for the detection of the antibody or antigen-binding fragment thereof. In particular embodiments, the label is an electrochemiluminescent ruthenium or iridium complex. In particular embodiments, the electrochemiluminescent ruthenium complex is a negatively charged electrochemiluminescent ruthenium complex. In particular embodiments, the label is a negatively charged electrochemiluminescent ruthenium complex which is present in the antigen with a stoichiometry of 1:1 to 15:1. In particular embodiments the stoichiometry is 2:1, 2.5:1, 3:1, 5:1, 10:1, or 15:1.

In particular embodiments, the antibody of the third aspect binds to the RBD of the spike protein of SARS-CoV-2 virus wildtype and mutant strains (variants).

In a fourth aspect, the present invention relates to an antibody or an antigen-binding fragment thereof, which
a) comprises CDR-H1, CDR-H2, CDR-H3, CDR-L1, CDR-L2 and CDR-L3 according to SEQ ID NO: 33, 34, 35, 36, 37, and 38, respectively,
b) binds to the same epitope as an antibody comprising CDR-H1, CDR-H2, CDR-H3, CDR-L1, CDR-L2 and CDR-L3 according to SEQ ID NO: 33, 34, 35, 36, 37, and 38, respectively,
or
c) which competes for binding to the RBD of the spike protein of SARS-CoV-2 virus with an antibody comprising CDR-H1, CDR-H2, CDR-H3, CDR-L1, CDR-L2 and CDR-L3 according to SEQ ID NO: 33, 34, 35, 36, 37, and 38, respectively.

In particular embodiments, the antibody or antigen-binding fragment thereof comprises CDRs comprising the sequences specifically recited above, i.e. without any amino acid variation.

In particular embodiments, the antibody or antigen-binding fragment thereof comprises one or more CDRs with sequence variations of the sequences recited above. In particular embodiments, the sequence variation comprises 1 or 2, in particular 1, amino acid alteration. In particular embodiments the 1 or 2 amino acids alterations are independently of each other amino acid deletions, amino acid additions, or amino acid substitutions. In particular embodiments, the amino acid substitution is a conservative amino acid substitution.

In particular embodiments, the antibody or antigen-binding fragment of the fourth aspect further
a) comprises FR-H1, FR-H2, FR-H3, FR-H4, FR-L1, FR-L2, FR-L3, and FR-L4 according to SEQ ID NO: 39, 40, 41, 42, 43, 44, 45, and 46, respectively,
b) binds to the same epitope as an antibody comprising FR-H1, FR-H2, FR-H3, FR-H4, FR-L1, FR-L2, FR-L3, and FR-L4 according to SEQ ID NO: 39, 40, 41, 42, 43, 44, 45, and 46, respectively,
or
c) competes for binding to the RBD of the spike protein of SARS-CoV-2 virus with an antibody comprising FR-H1, FR-H2, FR-H3, FR-H4, FR-L1, FR-L2, FR-L3, and FR-L4 according to SEQ ID NO: 39, 40, 41, 42, 43, 44, 45, and 46, respectively.

In particular embodiments, the antibody or antigen-binding fragment thereof comprises FRs comprising the sequences specifically recited above, i.e. without any amino acid variation.

In particular embodiments, the antibody or antigen-binding fragment thereof comprises one or more FRs with sequence variations of the sequences recited above. In particular embodiments, the sequence variation comprises up to 5, in particular 1, 2, 3, 4, or 5 amino acid alteration. In particular embodiments the up to 5, in particular 1, 2, 3, 4, or 5, amino acids alterations are independently of each other amino acid deletions, amino acid additions, or amino acid substitutions. In particular embodiments, the amino acid substitution is a conservative amino acid substitution.

In particular embodiments, the antibody or antigen-binding fragment of the fourth aspect
a) comprises a heavy chain variable domain having an amino acid sequence according to SEQ ID NO: 47 and a light chain variable domain having an amino acid sequence according to SEQ ID NO: 48,
b) binds to the same epitope as an antibody comprising a heavy chain variable domain having an amino acid sequence according to SEQ ID NO: 47 and a light chain variable domain having an amino acid sequence according to SEQ ID NO: 48,
or
c) competes for binding to the RBD of the spike protein of SARS-CoV-2 virus with an antibody comprising a heavy chain variable domain having an amino acid sequence according to SEQ ID NO: 47 and a light chain variable domain having an amino acid sequence according to SEQ ID NO: 48.

In particular embodiments, the antibody or antigen-binding fragment thereof comprises heavy chain variable domain and light chain variable domain comprising the sequences specifically recited above, i.e. without any amino acid variation.

In particular embodiments, the antibody or antigen-binding fragment thereof comprises heavy chain variable domain and light chain variable domain with sequence variations of the sequences recited above. In particular embodiments, the variant sequence is at least 85% identical to the sequences specifically recited above. In one further embodiment, the identity is at least 90%. In a further embodiment the identity is at least 95% in particular at least 98%.

In particular embodiments, the antibody or antigen-binding fragment thereof binds to the RBD of the spike protein of SARS-CoV-2 virus
a) with an association rate constant (kₐ) of more than 2.0E+06 M⁻¹s⁻¹, as determined by surface plasmon resonance,
and/or
b) with a dissociation rate constant (k_{d}) of less than 3.0E-03 s⁻¹, as determined by surface plasmon resonance,
and/or
c) with a half-life time of t/2diss of 4 minutes or more, as determined by surface plasmon resonance,
and/or
d) with a 1:1 or 1:2 stoichiometry.

In particular embodiments, the antibody has an association rate constant (kₐ) of more than 2.5E+06 M⁻¹s. In particular embodiments, the antibody has an association rate constant (kₐ) of more than 2.7E+06 M⁻¹s⁻¹, in particular of more than 3.0E+06 M⁻¹s⁻¹. In particular embodiments, the antibody has an association rate constant (kₐ) of more than 3.3E+06 M⁻¹s⁻¹.

In particular embodiments, the antibody has a dissociation rate constant (k_{d}) of less than 5.0E-03 s⁻¹, in particular of less than 4.5E-03 s⁻¹, in particular of less than 4.0E-03 s⁻¹, in particular of less than 3.5E-03 s⁻¹. 3.0E-03 s⁻¹, in particular of less than 2.7E-03 s⁻¹. In particular embodiments, the antibody has a dissociation rate constant (k_{d}) of less than 2.6E-03 s⁻¹, in particular of less than 1.1E-03 s⁻¹.

In particular embodiments, the antibody has an antibody/antigen complex half-life time of 4 minutes or more, t/2diss of 6 minutes or more, in particular of t/2diss of 11 minutes or more.

In particular embodiments, the antibody has an association rate constant (kₐ) of 3.0E+06 M⁻¹s⁻¹ and a dissociation rate constant (k_{d}) of 2.6E-03 s⁻¹. In particular embodiments, the antibody has an antibody/antigen complex half-life time of t/2diss of 4 min. In particular embodiments, the antibody has an association rate constant (kₐ) of 3.0E+06 M⁻¹s⁻¹ and a dissociation rate constant (k_{d}) of 2.6E-03 s⁻¹ and an antibody/antigen complex half-life time of t/2diss of 4 min.

In embodiments, the antibody or antigen-binding fragment of the present invention is an isolated antibody or antigen-binding fragment. Thus, the antibody or antigen-binding fragment is an antibody or antigen-binding fragment, which has been purified. Purification of an antibody can be achieved by methods well-known in the art such as Size Exclusion Chromatography (SEC). Accordingly, the antibody or antigen-binding fragment shall have been isolated from the cells in which the antibody was produced. In some embodiments, an isolated antibody or antigen-binding fragment is purified to greater than 70% by weight of antibody as determined by, for example, the Lowry method, and in some embodiments, to greater than 80%, 90%, 95%, 96%, 97%, 98% or 99% by weight. In one preferred embodiment the isolated antibody or antigen-binding fragment according to the present invention is purified to greater than 90% purity as determined by SDS-PAGE under reducing conditions using Coomassie blue staining for protein detection.

In embodiments, the antibody or antigen-binding fragment thereof is a naked antibody or naked antigen-binding fragment. In embodiments, the antibody or antigen-binding fragment thereof further comprises a tag or a label. In particular embodiments, the tag allows to bind the antibody or antigen-binding fragment thereof directly or indirectly to a solid phase. In particular embodiments, the tag is a partner of a bioaffine binding pair. In particular embodiments, the tag is selected from the group consisting of biotin, digoxin, hapten, or complementary oligonucleotide sequences (in particular complementary LNA sequences). In particular embodiments, the tag is biotin.

In particular embodiments, the label allows for the detection of the antibody or antigen-binding fragment thereof. In particular embodiments, the label is an electrochemiluminescent ruthenium or iridium complex. In particular embodiments, the electrochemiluminescent ruthenium complex is a negatively charged electrochemiluminescent ruthenium complex. In particular embodiments, the label is a negatively charged electrochemiluminescent ruthenium complex which is present in the antigen with a stoichiometry of 1:1 to 15:1. In particular embodiments the stoichiometry is 2:1, 2.5:1, 3:1, 5:1, 10:1, or 15:1.

In particular embodiments, the antibody of the fourth aspect binds to the RBD of the spike protein of SARS-CoV-2 virus wildtype and mutant strains (variants).

In a fifth aspect, the present invention relates to an isolated antibody or an antigen-binding fragment thereof, which
a) comprises CDR-H1, CDR-H2, CDR-H3, CDR-L1, CDR-L2 and CDR-L3 according to SEQ ID NO: 49, 50, 51, 52, 53, and 54, respectively,
b) binds to the same epitope as an antibody comprising CDR-H1, CDR-H2, CDR-H3, CDR-L1, CDR-L2 and CDR-L3 according to SEQ ID NO: 49, 50, 51, 52, 53, and 54, respectively,
   or
c) competes for binding to the RBD of the spike protein of SARS-CoV-2 virus with an antibody comprising CDR-H1, CDR-H2, CDR-H3, CDR-L1, CDR-L2 and CDR-L3 according to SEQ ID NO: 49, 50, 51, 52, 53, and 54, respectively.

In particular embodiments, the antibody or antigen-binding fragment thereof comprises CDRs comprising the sequences specifically recited above, i.e. without any amino acid variation.

In particular embodiments, the antibody or antigen-binding fragment thereof comprises one or more CDRs with sequence variations of the sequences recited above. In particular embodiments, the sequence variation comprises 1 or 2, in particular 1, amino acid alteration. In particular embodiments the 1 or 2 amino acids alterations are independently of each other amino acid deletions, amino acid additions, or amino acid substitutions. In particular embodiments, the amino acid substitution is a conservative amino acid substitution.

In particular embodiments, the antibody or antigen-binding fragment of the second aspect further
a) comprises FR-H1, FR-H2, FR-H3, FR-H4, FR-L1, FR-L2, FR-L3 and FR-L4 according to SEQ ID NO: 55, 56, 57, 58, 59, 60, 61 and 62, respectively,
b) binds to the same epitope as an antibody comprising FR-H1, FR-H2, FR-H3, FR-H4, FR-L1, FR-L2, FR-L3 and FR-L4 according to SEQ ID NO: 55, 56, 57, 58, 59, 60, 61 and 62, respectively,
or
c) competes for binding to the RBD of the spike protein of SARS-CoV-2 virus with an antibody comprising FR-H1, FR-H2, FR-H3, FR-H4, FR-L1, FR-L2, FR-L3 and FR-L4 according to SEQ ID NO: 55, 56, 57, 58, 59, 60, 61 and 62, respectively.

In particular embodiments, the antibody or antigen-binding fragment thereof comprises FRs comprising the sequences specifically recited above, i.e. without any amino acid variation.

In particular embodiments, the antibody or antigen-binding fragment thereof comprises one or more FRs with sequence variations of the sequences recited above. In particular embodiments, the sequence variation comprises up to 5, in particular 1, 2, 3, 4, or 5 amino acid alteration. In particular embodiments the up to 5, in particular 1, 2, 3, 4, or 5, amino acids alterations are independently of each other amino acid deletions, amino acid additions, or amino acid substitutions. In particular embodiments, the amino acid substitution is a conservative amino acid substitution.

In particular embodiments, the antibody or antigen-binding fragment of the second aspect
a) comprises a heavy chain variable domain having an amino acid sequence according to SEQ ID NO: 63 and a light chain variable domain having an amino acid sequence according to SEQ ID NO: 64
b) binds to the same epitope as an antibody comprising a heavy chain variable domain having an amino acid sequence according to SEQ ID NO: 63 and a light chain variable domain having an amino acid sequence according to SEQ ID NO: 64
or
c) competes for binding to the RBD of the spike protein of SARS-CoV-2 virus with an antibody comprising a heavy chain variable domain having an amino acid sequence according to SEQ ID NO: 63 and a light chain variable domain having an amino acid sequence according to SEQ ID NO: 64.

In particular embodiments, the antibody or antigen-binding fragment thereof comprises heavy chain variable domain and light chain variable domain comprising the sequences specifically recited above, i.e. without any amino acid variation.

In particular embodiments, the antibody or antigen-binding fragment thereof comprises heavy chain variable domain and light chain variable domain with sequence variations of the sequences recited above. In particular embodiments, the variant sequence is at least 85% identical to the sequences specifically recited above. In one further embodiment, the identity is at least 90%. In a further embodiment the identity is at least 95% in particular at least 98%.

In particular embodiments, the antibody or antigen-binding fragment thereof binds to the RBD of the spike protein of SARS-CoV-2 virus
a) with an association rate constant (kₐ) of more than 2.0E+06 M⁻¹s⁻¹, as determined by surface plasmon resonance,
and/or
b) with a dissociation rate constant (k_{d}) of less than 3.0E-03 s⁻¹, as determined by surface plasmon resonance,
and/or
c) with a half-life time of t/2diss of 4 minutes or more, as determined by surface plasmon resonance,
and/or
d) with a 1:1 or 1:2 stoichometry.

In particular embodiments, the antibody has an association rate constant (kₐ) of more than 2.5E+06 M⁻¹s. In particular embodiments, the antibody has an association rate constant (kₐ) of more than 2.7E+06 M⁻¹s⁻¹, in particular of more than 3.0E+06 M⁻¹s⁻¹. In particular embodiments, the antibody has an association rate constant (kₐ) of more than 3.3E+06 M⁻¹s⁻¹.

In particular embodiments, the antibody has a dissociation rate constant (k_{d}) of less than 5.0E-03 s⁻¹, in particular of less than 4.5E-03 s⁻¹, in particular of less than 4.0E-03 s⁻¹, in particular of less than 3.5E-03 s⁻¹, 3.0E-03 s⁻¹, in particular of less than 2.7E-03 s⁻¹. In particular embodiments, the antibody has a dissociation rate constant (k_{d}) of less than 2.6E-03 s⁻¹, in particular of less than 1.1E-03 s⁻¹.

In particular embodiments, the antibody has an antibody/antigen complex half-life time of t/2diss of 4 minutes or more, t/2diss of 6 minutes or more, in particular of t/2diss of 11 minutes or more.

In particular embodiments, the antibody has an association rate constant (kₐ) of 2.5E+06 M⁻¹s⁻¹ and a dissociation rate constant (k_{d}) of 1.9E-03 s⁻¹. In particular embodiments, the antibody has an antibody/antigen complex half-life time of t/2diss of 6 min. In particular embodiments, the antibody has an association rate constant (kₐ) of 2.5E+06 M⁻¹s⁻¹ and a dissociation rate constant (k_{d}) of 1.9E-03 s⁻¹ and an antibody/antigen complex half-life time of t/2diss of 6 min.

In embodiments, the antibody or antigen-binding fragment of the present invention is an isolated antibody or antigen-binding fragment. Thus, the antibody or antigen-binding fragment is an antibody or antigen-binding fragment, which has been purified. Purification of an antibody can be achieved by methods well-known in the art such as Size Exclusion Chromatography (SEC). Accordingly, the antibody or antigen-binding fragment shall have been isolated from the cells in which the antibody was produced. In some embodiments, an isolated antibody or antigen-binding fragment is purified to greater than 70% by weight of antibody as determined by, for example, the Lowry method, and in some embodiments, to greater than 80%, 90%, 95%, 96%, 97%, 98% or 99% by weight. In one preferred embodiment the isolated antibody or antigen-binding fragment according to the present invention is purified to greater than 90% purity as determined by SDS-PAGE under reducing conditions using Coomassie blue staining for protein detection.

In embodiments, the antibody or antigen-binding fragment thereof is a naked antibody or naked antigen-binding fragment. In embodiments, the antibody or antigen-binding fragment thereof further comprises a tag or a label. In particular embodiments, the tag allows to bind the antibody or antigen-binding fragment thereof directly or indirectly to a solid phase. In particular embodiments, the tag is a partner of a bioaffine binding pair. In particular embodiments, the tag is selected from the group consisting of biotin, digoxin, hapten, or complementary oligonucleotide sequences (in particular complementary LNA sequences). In particular embodiments, the tag is biotin.

In particular embodiments, the label allows for the detection of the antibody or antigen-binding fragment thereof. In particular embodiments, the label is an electrochemiluminescent ruthenium or iridium complex. In particular embodiments, the electrochemiluminescent ruthenium complex is a negatively charged electrochemiluminescent ruthenium complex. In particular embodiments, the label is a negatively charged electrochemiluminescent ruthenium complex which is present in the antigen with a stoichiometry of 1:1 to 15:1. In particular embodiments the stoichiometry is 2:1, 2.5:1, 3:1, 5:1, 10:1, or 15:1.

In an sixth aspect, the present invention relates to a kit comprising at least one antibody selected from the group of antibodies as described above for the first aspect, the second aspect, the third aspect, the fourth aspect, or the fifth aspect of the present invention. Accordingly, in embodiments, the kit may comprise the antibody as described above for the first aspect of the present invention. In further embodiments, the kit may comprise the antibody as described above for the second aspect of the present invention. In further embodiments, the kit may comprise the antibody as described above for the third aspect of the present invention. In further embodiments, the kit may comprise the antibody as described above for the fourth aspect of the present invention. In further embodiments, the kit may comprise the antibody as described above for the fifth aspect of the present invention.

In particular embodiments, the kit further comprises a second antibody selected from the group of antibodies as described above for the first aspect, the second aspect, the third aspect, the fourth aspect, or the fifth aspect of the present invention.

Accordingly, in embodiments, the kit may comprise the antibody as described above for the first aspect and the antibody as described above for the second aspect of the present invention. In further embodiments, the kit may comprise the antibody as described above for the first aspect and the antibody as described above for the third aspect of the present invention. In further embodiments, the kit may comprise the antibody as described above for the first aspect and the antibody as described above for the fourth aspect of the present invention. In further embodiments, the kit may comprise the antibody as described above for the first aspect and the antibody as described above for the fifth aspect of the present invention. In further embodiments, the kit may comprise the antibody as described above for the second aspect and the antibody as described above for the third aspect of the present invention. In further embodiments, the kit may comprise the antibody as described above for the second aspect and the antibody as described above for the fourth aspect of the present invention. In further embodiments, the kit may comprise the antibody as described above for the second aspect and the antibody as described above for the fifth aspect of the present invention. In further embodiments, the kit may comprise the antibody as described above for the third aspect and the antibody as described above for the fourth aspect of the present invention. In further embodiments, the kit may comprise the antibody as described above for the third aspect and the antibody as described above for the fifth aspect of the present invention. In further embodiments, the kit may comprise the antibody as described above for the fourth aspect and the antibody as described above for the fifth aspect of the present invention.

In particular embodiments, the kit further comprises a third antibody selected from the group of antibodies as described above for the first aspect, the second aspect, the third aspect, the fourth aspect, or the fifth aspect of the present invention. Accordingly, in embodiments, the kit may comprise the antibody as described above for the first aspect, the antibody as described above for the second aspect, and the antibody as described above for the third aspect of the present invention. In further embodiments, the kit may comprise the antibody as described above for the first aspect, the antibody as described above for the second aspect, and the antibody as described above for the fourth aspect of the present invention. In particular embodiments the kit may comprise any combination of three antibodies according to the first aspect, the second aspect, the third aspect, the fourth aspect of the fifth aspect of the present invention.

In a seventh aspect, the present invention relates to a nucleic acid encoding an antibody selected from the group of antibodies as described above for the first aspect, the second aspect, the third aspect, the fourth aspect, or the fifth aspect of the present invention.

In an eight aspect, the present invention relates to a host cell comprising the nucleic acid as described above for the seventh aspect of the present invention, and/or producing an antibody as described above for the first aspect, the second, the third aspect, the fourth aspect, or the fifth aspect of the present invention.

In a preferred embodiment, the host cell is a hybridoma cell. Moreover, the host cell may be any kind of cellular system, which can be engineered to generate the antibodies according to the current invention. For example, the host cell may be an animal cell, in particular a mammalian cell. In one embodiment HEK293 (human embryonic kidney cells) such as HEK 293-F cells as used in the Examples section, or CHO (Chinese hamster ovary) cells are used as host cells. In another embodiment, the host cell is a non-human animal or mammalian cell.

The host cell preferably comprises at least one polynucleotide encoding for the antibody of the present invention, or fragment thereof. In particular embodiments, the host cell comprises the nucleic acid of the seventh aspect of the present invention. In particular, the host cell comprises at least one polynucleotide encoding for the light chain of the antibody of the present invention and at least one polynucleotide encoding the heavy chain of the antibody of the present invention. Said polynucleotide(s) shall be operably linked to a suitable promoter.

In an ninth aspect, the present invention relates to a composition comprising at least one antibody selected from the group of antibodies as described above for the first aspect, the second aspect, the third aspect, the fourth aspect, or the fifth aspect of the present invention. Accordingly, in embodiments, the composition may comprise the antibody as described above for the first aspect of the present invention. In further embodiments, the composition may comprise the antibody as described above for the second aspect of the present invention. In further embodiments, the composition may comprise the antibody as described above for the third aspect of the present invention. In further embodiments, the composition may comprise the antibody as described above for the fourth aspect of the present invention. In further embodiments, the composition may comprise the antibody as described above for the fifth aspect of the present invention.

In particular embodiments, the composition further comprises a second antibody selected from the group of antibodies as described above for the first aspect, the second aspect, the third aspect, the fourth aspect, or the fifth aspect of the present invention.

Accordingly, in embodiments, the composition may comprise the antibody as described above for the first aspect and the antibody as described above for the second aspect of the present invention. In further embodiments, the composition may comprise the antibody as described above for the first aspect and the antibody as described above for the third aspect of the present invention. In further embodiments, the composition may comprise the antibody as described above for the first aspect and the antibody as described above for the fourth aspect of the present invention. In further embodiments, the composition may comprise the antibody as described above for the first aspect and the antibody as described above for the fifth aspect of the present invention. In further embodiments, the composition may comprise the antibody as described above for the second aspect and the antibody as described above for the third aspect of the present invention. In further embodiments, the composition may comprise the antibody as described above for the second aspect and the antibody as described above for the fourth aspect of the present invention. In further embodiments, the composition may comprise the antibody as described above for the second aspect and the antibody as described above for the fifth aspect of the present invention. In further embodiments, the composition may comprise the antibody as described above for the third aspect and the antibody as described above for the fourth aspect of the present invention. In further embodiments, the composition may comprise the antibody as described above for the third aspect and the antibody as described above for the fifth aspect of the present invention. In further embodiments, the composition may comprise the antibody as described above for the fourth aspect and the antibody as described above for the fifth aspect of the present invention.

In particular embodiments, the composition further comprises a third antibody selected from the group of antibodies as described above for the first aspect, the second aspect, the third aspect, the fourth aspect, or the fifth aspect of the present invention. Accordingly, in embodiments, the composition may comprise the antibody as described above for the first aspect, the antibody as described above for the second aspect, and the antibody as described above for the third aspect of the present invention. In further embodiments, the composition may comprise the antibody as described above for the first aspect, the antibody as described above for the second aspect, and the antibody as described above for the fourth aspect of the present invention. In further embodiments, the composition may comprise the antibody as described above for the second aspect, the antibody as described above for the third aspect, and the antibody as described above for the fourth aspect of the present invention. In further embodiments, the composition may comprise the antibody as described above for the first aspect, the antibody as described above for the third aspect, and the antibody as described above for the fourth aspect of the present invention. In further embodiments, the composition may comprise the antibody as described above for the first aspect, the antibody as described above for the second aspect, and the antibody as described above for the fourth aspect of the present invention. In particular embodiments the kit may comprise any combination of three antibodies according to the first aspect, the second aspect, the third aspect, the fourth aspect of the fifth aspect of the present invention.

In particular embodiments, the composition is a diagnostic composition. Accordingly, in particular embodiments, the composition is for diagnostic use.

In a tenth aspect, the present invention relates to the use of an antibody or antigen binding fragment of the first aspect, the second aspect, the third aspect, the fourth aspect, or the fifth aspect of the present invention, or the kit of the sixth aspect of the present invention or the composition of the ninth aspect of the present invention, for an in vitro immunoassay. In particular embodiments, the immunoassay is an heterologous immunoassay.

In an eleventh aspect, the present invention relates to an in vitro method for detecting the presence of SARS-CoV-2 virus in a sample obtained from a patient, comprising
a) incubating the sample with at least one antibody or antibody binding fragment thereof which binds to the RBD of the Spike protein of SARS-CoV-2, thereby generating a complex between the least one antibody or antibody binding fragment and the RBD of the Spike protein of SARS-CoV-2,
b) optionally immobilizing the formed complexes to a solid phase, in particular to microparticles, and
c) detecting the complex formed in step a), thereby detecting the presence of SARS-CoV-2 virus in the sample.

In an embodiment, the aforementioned method does not encompass the drawing of the sample from the subject. Rather, the sample which has been obtained from the subject (e.g. under supervision of the attending physician) is provided. For example, the sample can be provided by delivering the sample to a laboratory, which carries out detecting the presence of SARS-CoV-2 virus in said sample.

In particular embodiments, the at least one antibody or antibody binding fragment is an antibody or antibody binding fragment of the first aspect, the second aspect, the third aspect, the fourth aspect and/or the fifth aspect of the present invention.

In embodiments, the sample is incubated in step a) with the antibody as described above for the first aspect of the present invention. In further embodiments, the sample is incubated with the antibody as described above for the second aspect of the present invention. In further embodiments, the sample is incubated with the antibody as described above for the third aspect of the present invention. In further embodiments, the sample is incubated with the antibody as described above for the fourth aspect of the present invention. In further embodiments, the sample is incubated with the antibody as described above for the fifth aspect of the present invention.

In particular embodiments, the sample is further incubated in step a) with a second antibody selected from the group of antibodies as described above for the first aspect, the second aspect, the third aspect, the fourth aspect, or the fifth aspect of the present invention.

In particular embodiments, in step a) the sample is incubated with two antibodies, binding to the RBD of the Spike protein of SARS-CoV-2. As obvious to the skilled artisan, the sample can be contacted with the first and the second antibody in any desired order, i.e. first antibody first and then the second antibody or second antibody first and then the first antibody, or simultaneously, for a time and under conditions sufficient to form a first anti-SARS-CoV-2 RBD-antibody/SARS-CoV-2 RDB-antigen/second anti-SARS-CoV-2 RBD-antibody complex. As the skilled artisan will readily appreciate it is nothing but routine experimentation to establish the time and conditions that are appropriate or that are sufficient for the formation of a complex either between the specific anti SARS-CoV-2 RBD antibody and the SARS-CoV-2 RBD-antigen/analyte (=anti-SARS-CoV-2 S-complex) or the formation of the secondary, or sandwich complex comprising the first antibody anti-SARS-CoV-2 RBD-antibody, SARS-CoV-2 RBD-antigen (the analyte) and the second anti-SARS-CoV-2 RBD-antibody(=first anti-SARS-CoV-2 RBD-antibody/SARS-CoV-2 RBD-antigen/second anti-SARS-CoV-2 RBD-antibody complex).

The detection of the anti-SARS-CoV-2 RBD-antibody/SARS-CoV-2 RBD-antigen complex can be performed by any appropriate means. The detection of the first anti-SARS-CoV-2 RBD-antibody/SARS-CoV-2 RBD-antigen/second anti-SARS-CoV-2 RBD-antibody complex can be performed by any appropriate means. The person skilled in the art is absolutely familiar with such means/methods.

Accordingly, in embodiments, the sample is incubated in step a) with the antibody as described above for the first aspect and the antibody as described above for the second aspect of the present invention. In further embodiments, the sample is incubated with the antibody as described above for the first aspect and the antibody as described above for the third aspect of the present invention. In further embodiments, the sample is incubated with the antibody as described above for the first aspect and the antibody as described above for the fourth aspect of the present invention. In further embodiments, the sample is incubated with the antibody as described above for the first aspect and the antibody as described above for the fifth aspect of the present invention. In further embodiments, the sample is incubated with the antibody as described above for the second aspect and the antibody as described above for the third aspect of the present invention. In further embodiments, the sample is incubated with the antibody as described above for the second aspect and the antibody as described above for the fourth aspect of the present invention. In further embodiments, the sample is incubated with the antibody as described above for the second aspect and the antibody as described above for the fifth aspect of the present invention. In further embodiments, the sample is incubated with the antibody as described above for the third aspect and the antibody as described above for the fourth aspect of the present invention. In further embodiments, the sample is incubated with the antibody as described above for the third aspect and the antibody as described above for the fifth aspect of the present invention. In further embodiments, the sample is incubated with the antibody as described above for the fourth aspect and the antibody as described above for the fifth aspect of the present invention.

In particular embodiments, the sample is further incubated in step a) with a third antibody selected from the group of antibodies as described above for the first aspect, the second aspect, the third aspect, the fourth aspect, or the fifth aspect of the present invention. Accordingly, in embodiments, the sample is incubated with the antibody as described above for the first aspect, the antibody as described above for the second aspect, and the antibody as described above for the third aspect of the present invention. In further embodiments, the sample is incubated with the antibody as described above for the first aspect, the antibody as described above for the second aspect, and the antibody as described above for the fourth aspect of the present invention. In further embodiments, the sample is incubated in step a) with the antibody as described above for the second aspect, the antibody as described above for the third aspect, and the antibody as described above for the fourth aspect of the present invention. In further embodiments, the sample is incubated with the antibody as described above for the first aspect, the antibody as described above for the third aspect, and the antibody as described above for the fourth aspect of the present invention. In further embodiments, the sample is incubated with the antibody as described above for the first aspect, the antibody as described above for the second aspect, and the antibody as described above for the fourth aspect of the present invention. In particular embodiments the sample is incubated with any combination of three antibodies according to the first aspect, the second aspect, the third aspect, the fourth aspect of the fifth aspect of the present invention.

In embodiments, the first antibody is capable of immobilizing on a solid phase and the second antibody is labeled with a detectable label. In embodiments, the detectable label is a luminescent dye, in particular a chemiluminescent dye or an electrochemiluminescent dye. In embodiments, the antibody capable of immobilizing on a solid phase is tagged, in particular with a partner of a bioaffine binding pair, in particular biotin or a complementary LNA sequences.

In embodiments, the first antibody is labeled with a detectable label and the second antibody is capable of immobilizing on a solid phase. In embodiments, the detectable label is a luminescent dye, in particular a chemiluminescent dye or an electrochemiluminescent dye. In embodiments, the antibody capable of immobilizing on a solid phase is tagged, in particular with a partner of a bioaffine binding pair, in particular biotin or a complementary LNA sequences.

In embodiments, the first antibody is capable of immobilizing on a solid phase and the second antibody is labeled with a detectable label, and the third antibody is labeled with a detectable label. In embodiments, the detectable label is a luminescent dye, in particular a chemiluminescent dye or an electrochemiluminescent dye. In embodiments, the antibody capable of immobilizing on a solid phase is tagged, in particular with a partner of a bioaffine binding pair, in particular biotin or a complementary LNA sequences.

In embodiments, the first antibody is labeled with a detectable label and the second antibody is capable of immobilizing on a solid phase, and the third antibody is labeled with a detectable label. In embodiments, the detectable label is a luminescent dye, in particular a chemiluminescent dye or an electrochemiluminescent dye. In embodiments, the antibody capable of immobilizing on a solid phase is tagged, in particular with a partner of a bioaffine binding pair, in particular biotin or a complementary LNA sequences.

In embodiments, the method is an enzyme-linked immunoassay (ELISA) or electrochemiluminescence immunoassay (ECLIA) or radioimmunoassay (RIA). In particular embodiments, the method is an ELICA method.

In particular embodiments, the sample of the patient is a fluid sample, in particular a fluid body sample. In particular embodiments, the sample is selected from the group consisting of nasopharyngeal swab, oropharyngeal swab, sputum, saliva, whole blood, serum, or plasma. In particular embodiments, the sample is selected from the group consisting of nasopharyngeal swab, oropharyngeal swab, sputum, saliva. In particular embodiments, the sample is a nasopharyngeal swab or oropharyngeal swab. In embodiments, the sample is an in vitro sample, i.e. it will be analyzed in vitro and not transferred back into the body.

In particular embodiments, the patient is a laboratory animal, a domestic animal or a primate. In particular embodiments, the patient is a human patient.

In particular embodiments, the method for detecting the presence of SARS-CoV-2 virus in a sample obtained from a patient according to the eleventh aspect of the present invention, is capable of detecting also mutants of SARS-CoV-2 (variants).

In further embodiments, the present invention relates to the following items:
1. An (isolated) monoclonal antibody or antigen-binding fragment thereof that binds to the RBD of the Spike protein of SARS-CoV-2 virus
   a) with an association rate constant (kₐ) of more than 2.5E+06 M⁻¹s⁻¹, as determined by surface plasmon resonance, and/or
   b) with a dissociation rate constant (k_{d}) of less than 5.0E-03 s⁻¹, as determined by surface plasmon resonance, and/or
   c) with a half-life time of t/2diss of 4 minutes or more, as determined by surface plasmon resonance, and/or
   d) with a 1:1 or 1:2 stoichometry.
2. The isolated monoclonal antibody or antigen-binding fragment of item 1, which
   a) comprises CDR-H1, CDR-H2, CDR-H3, CDR-L1, CDR-L2 and CDR-L3 according to SEQ ID NO: 1, 2, 3, 4, 5, and 6, respectively,
   b) binds to the same epitope as an antibody comprising CDR-H1, CDR-H2, CDR-H3, CDR-L1, CDR-L2 and CDR-L3 according to SEQ ID NO: 1, 2, 3, 4, 5, and 6, respectively, or
   c) competes for binding to the RBD of the Spike protein of SARS-CoV-2 virus with an antibody comprising CDR-H1, CDR-H2, CDR-H3, CDR-L1, CDR-L2 and CDR-L3 according to SEQ ID NO: 1, 2, 3, 4, 5, and 6, respectively.
3. The isolated monoclonal antibody or antigen-binding fragment of item 2, which
   a) comprises FR-H1, FR-H2, FR-H3, FR-H4, FR-L1, FR-L2, FR-L3 and FR-L4 according to SEQ ID NO: 7, 8, 9, 10, 11, 12, 13, and 14, respectively,
   b) binds to the same epitope as an antibody comprising FR-H1, FR-H2, FR-H3, FR-H4, FR-L1, FR-L2, FR-L3 and FR-L4 according to SEQ ID NO: 7, 8, 9, 10, 11, 12, 13, and 14, respectively, or
   c) competes for binding to the RBD of the Spike protein of SARS-CoV-2 virus with an antibody comprising FR-H1, FR-H2, FR-H3, FR-H4, FR-L1, FR-L2, FR-L3 and FR-L4 according to SEQ ID NO: 7, 8, 9, 10, 11, 12, 13, and 14, respectively.
4. The isolated monoclonal antibody or antigen-binding fragment of any of items 1 to 3, which
   a) comprises a heavy chain variable domain having an amino acid sequence according to SEQ ID NO: 15 and a light chain variable domain having an amino acid sequence according to SEQ ID NO: 16
   b) binds to the same epitope as an antibody comprising a heavy chain variable domain having an amino acid sequence according to SEQ ID NO: 15 and a light chain variable domain having an amino acid sequence according to SEQ ID NO: 16
      or
   c) competes for binding to the RBD of the Spike protein of SARS-CoV-2 virus with an antibody comprising a heavy chain variable domain having an amino acid sequence according to SEQ ID NO: 15 and a light chain variable domain having an amino acid sequence according to SEQ ID NO: 16.
5. The isolated monoclonal antibody or antigen-binding fragment of item 1, which
   a) comprises CDR-H1, CDR-H2, CDR-H3, CDR-L1, CDR-L2 and CDR-L3 according to SEQ ID NO: 17, 18, 19, 20, 21, and 22, respectively,
   b) binds to the same epitope as an antibody comprising CDR-H1, CDR-H2, CDR-H3, CDR-L1, CDR-L2 and CDR-L3 according to SEQ ID NO: 17, 18, 19, 20, 21, and 22, respectively,
   or
   c) which competes for binding to the RBD of the Spike protein of SARS-CoV-2 virus with an antibody comprising CDR-H1, CDR-H2, CDR-H3, CDR-L1, CDR-L2 and CDR-L3 according to SEQ ID NO: 17, 18, 19, 20, 21 and 22, respectively.
6. The isolated monoclonal antibody or antigen-binding fragment of item 5, which
   a) comprises FR-H1, FR-H2, FR-H3, FR-H4, FR-L1, FR-L2, FR-L3, and FR-L4 according to SEQ ID NO: 23, 24, 25, 26, 27, 28, 29, and 30, respectively,
   b) binds to the same epitope as an antibody comprising FR-H1, FR-H2, FR-H3, FR-H4, FR-L1, FR-L2, FR-L3, and FR-L4 according to SEQ ID NO: 23, 24, 25, 26, 27, 28, 29, and 30, respectively,
   or
   c) competes for binding to the RBD of the Spike protein of SARS-CoV-2 virus with an antibody comprising FR-H1, FR-H2, FR-H3, FR-H4, FR-L1, FR-L2, FR-L3, and FR-L4 according to SEQ ID NO: 23, 24, 25, 26, 27, 28, 29, and 30, respectively.
7. The isolated monoclonal antibody or antigen-binding fragment of any of items 1, 5, or 6, which
   a) comprises a heavy chain variable domain having an amino acid sequence according to SEQ ID NO: 31 and a light chain variable domain having an amino acid sequence according to SEQ ID NO: 32,
   b) binds to the same epitope as an antibody comprising a heavy chain variable domain having an amino acid sequence according to SEQ ID NO: 31 and a light chain variable domain having an amino acid sequence according to SEQ ID NO: 32,
   or
   c) competes for binding to the RBD of the Spike protein of SARS-CoV-2 virus with an antibody comprising a heavy chain variable domain having an amino acid sequence according to SEQ ID NO: 31 and a light chain variable domain having an amino acid sequence according to SEQ ID NO: 32.
8. The isolated monoclonal antibody or antigen-binding fragment of item 1, which
   a) comprises CDR-H1, CDR-H2, CDR-H3, CDR-L1, CDR-L2 and CDR-L3 according to SEQ ID NO: 33, 34, 35, 36, 37, and 38, respectively,
   b) binds to the same epitope as an antibody comprising CDR-H1, CDR-H2, CDR-H3, CDR-L1, CDR-L2 and CDR-L3 according to SEQ ID NO: 33, 34, 35, 36, 37, and 38, respectively,
   or
   c) which competes for binding to the RBD of the Spike protein of SARS-CoV-2 virus with an antibody comprising CDR-H1, CDR-H2, CDR-H3, CDR-L1, CDR-L2 and CDR-L3 according to SEQ ID NO: 33, 34, 35, 36, 37, and 38, respectively.
9. The isolated monoclonal antibody or antigen-binding fragment of item 8, which
   a) comprises FR-H1, FR-H2, FR-H3, FR-H4, FR-L1, FR-L2, FR-L3, and FR-L4 according to SEQ ID NO: 39, 40, 41, 42, 43, 44, 45, and 46, respectively,
   b) binds to the same epitope as an antibody comprising FR-H1, FR-H2, FR-H3, FR-H4, FR-L1, FR-L2, FR-L3, and FR-L4 according to SEQ ID NO: 39, 40, 41, 42, 43, 44, 45, and 46, respectively,
   or
   c) competes for binding to the RBD of the Spike protein of SARS-CoV-2 virus with an antibody comprising FR-H1, FR-H2, FR-H3, FR-H4, FR-L1, FR-L2, FR-L3, and FR-L4 according to SEQ ID NO: 39, 40, 41, 42, 43, 44, 45, and 46, respectively.
10. The isolated monoclonal antibody or antigen-binding fragment of any of items 1, 8, or 9, which
   a) comprises a heavy chain variable domain having an amino acid sequence according to SEQ ID NO: 47 and a light chain variable domain having an amino acid sequence according to SEQ ID NO: 48,
   b) binds to the same epitope as an antibody comprising a heavy chain variable domain having an amino acid sequence according to SEQ ID NO: 47 and a light chain variable domain having an amino acid sequence according to SEQ ID NO: 48,
   or
   c) competes for binding to the RBD of the Spike protein of SARS-CoV-2 virus with an antibody comprising a heavy chain variable domain having an amino acid sequence according to SEQ ID NO: 47 and a light chain variable domain having an amino acid sequence according to SEQ ID NO: 48.
11. The isolated monoclonal antibody or antigen-binding fragment of item 1, which
   a) comprises CDR-H1, CDR-H2, CDR-H3, CDR-L1, CDR-L2 and CDR-L3 according to SEQ ID NO: 49, 50, 51, 52, 53, and 54, respectively,
   b) binds to the same epitope as an antibody comprising CDR-H1, CDR-H2, CDR-H3, CDR-L1, CDR-L2 and CDR-L3 according to SEQ ID NO: 49, 50, 51, 52, 53, and 54, respectively,
   or
   c) which competes for binding to the RBD of the Spike protein of SARS-CoV-2 virus with an antibody comprising CDR-H1, CDR-H2, CDR-H3, CDR-L1, CDR-L2 and CDR-L3 according to SEQ ID NO: 49, 50, 51, 52, 53, and 54, respectively.
12. The isolated monoclonal antibody or antigen-binding fragment of item 8, which
   a) comprises FR-H1, FR-H2, FR-H3, FR-H4, FR-L1, FR-L2, FR-L3, and FR-L4 according to SEQ ID NO: 55, 56, 57, 58, 59, 60, 61 and 62, respectively,
   b) binds to the same epitope as an antibody comprising FR-H1, FR-H2, FR-H3, FR-H4, FR-L1, FR-L2, FR-L3, and FR-L4 according to SEQ ID NO: 55, 56, 57, 58, 59, 60, 61 and 62, respectively,
   or
   c) competes for binding to the RBD of the Spike protein of SARS-CoV-2 virus with an antibody comprising FR-H1, FR-H2, FR-H3, FR-H4, FR-L1, FR-L2, FR-L3, and FR-L4 according to SEQ ID NO: 55, 56, 57, 58, 59, 60, 61 and 62, respectively.
13. The isolated monoclonal antibody or antigen-binding fragment of any of items 1, 8, or 9, which
   a) comprises a heavy chain variable domain having an amino acid sequence according to SEQ ID NO: 63 and a light chain variable domain having an amino acid sequence according to SEQ ID NO: 64,
   b) binds to the same epitope as an antibody comprising a heavy chain variable domain having an amino acid sequence according to SEQ ID NO: 63 and a light chain variable domain having an amino acid sequence according to SEQ ID NO: 64,
   or
   c) competes for binding to the RBD of the Spike protein of SARS-CoV-2 virus with an antibody comprising a heavy chain variable domain having an amino acid sequence according to SEQ ID NO: 63 and a light chain variable domain having an amino acid sequence according to SEQ ID NO: 64.
14. A kit comprising at least one antibody according to any of items 2 to 4, and optionally a second antibody according to any of items 5 to 7, optionally a third antibody according to any of items 8 to 10, and optionally a fourth antibody according to any of items 11 to 13.
15. A nucleic acid encoding an antibody as defined in any of items 1 to 13.
16. A host cell comprising the nucleic acid of item 15, and/or producing an antibody as defined in any of items 1 to 13.
17. A composition comprising the antibody as defined in any of items 1 to 13.
18. Use of the antibody according to any one of items 1 to 13, the kit of item 14, or the composition according to item 17 for an in vitro immunoassay.
19. An in vitro method for detecting the presence of SARS-CoV-2 virus in a sample obtained from a patient, comprising
   a) incubating the sample with at least one antibody or antibody binding fragment thereof which binds to the RBD of Spike protein of SARS-CoV-2, in particular with at least one antibody or antibody binding fragment thereof of any of items 1 to 13, thereby generating a complex between the antibody and the RBD of the Spike protein of SARS-CoV-2,
   b) optionally immobilizing the formed complexes to a solid phase, in particular to microparticles, and
   c) detecting the presence of SARS-CoV-2 virus in the sample.
20. The method according to item 19, wherein the sample of the patient is selected from the group consisting of nasopharyngeal swab, oropharyngeal swab, sputum, saliva, . . . .

The following examples and figures are provided to aid the understanding of the present invention, the true scope of which is set forth in the appended claims. It is understood that modifications can be made in the procedures set forth without departing from the spirit of the invention.

### EXAMPLES

### Example 1: Generation of Antibodies

For the generation of highly specific antibodies against the RBD of the SARS-CoV-2 Spike protein, we immunized New Zealand white rabbits and NMRI mice with RBD of the S1 subunit of the Spike glycoprotein and screened subsequently for RBD binding antibodies.

Immunogen: SARS-CoV-2 RBD (corresponding to amino acids at position 319-541 of the full-length spike protein, according to the sequence disclosed in https://www.uniprot.org/uniprot/PODTC2), expressed in HEK cells.

Screening Reagent: Biotinylated SARS-CoV-2 mSpike and RBD Protein (as described in: Amanat et al., A serological assay to detect SARS-CoV-2 seroconversion in humans, Nature Medicine, Vol. 26, 1033-1036 (2020)).

The immunization procedure resulted in various individual rabbit and mouse IgG clones reacting specifically with S1-RBD protein from SARS-CoV-2, but not with other coronaviruses (HKU-1, SARS-CoV-1, MERS and OC43—data not shown). Specificity of these RBD antibodies was demonstrated by ELISA assays and SPR Biacore analysis of the B cell supernatant and mouse hybridoma supernatant, respectively (not shown).

### Example 2: Antibody SPR Screening

The kinetic screening of the generated antibodies was performed at 37° C. on GE Healthcare BIAcore™ 8K+, 8K and B4000 instruments. A Biacore CM5 Series S sensor was mounted to the instrument and was preconditioned according to the manufacturer's instructions.

The system buffer was HBS ET pH 7.4, 10 mM HEPES, pH 7.4, 150 mM NaCl, 3 mM EDTA, 0.05% (w/v) Tween20. The system buffer was supplemented with 1 mg/mL CMD (Carboxymethyldextran, Fluka) and was used as sample buffer for the preparation of dilution series.

A rabbit or mouse specific antibody capture system was immobilized on the sensor surface using HBS-N pH 7.4 as a system buffer. A polyclonal goat anti-rabbit IgG Fc capture antibody GARbFc7 (Code-No. 111-005-046, Jackson Immuno Research) or a polyclonal goat anti-mouse Fcy capture antibody PAK<M-IgG(Fcy)>Z (Code-No. 115-005-071, Jackson Immuno Research) was amine coupled using the EDC/NHS-chemistry according to the to the manufacturer's instructions. 30 μg/mL capture antibody were used in 10 mM sodium acetate buffer. For the rabbit antibody capturing the solution was adjusted to pH 4.5, for the mouse capturing the solution was adjusted to pH 5. Capturing antibodies were immobilized with ligand densities of approximately 10000 RU-15000 RU. Free activated carboxyl groups were subsequently saturated with 1 M ethanolamine pH 8.5.

Flow cells 1 on all channels served as references on the 8K instruments. Using the B4000, the spots 2 and 4 were used as references. Each rabbit or mouse antibody solution was diluted in sample buffer and was injected at a 5 μl/min or 10 μL/min for 2 minutes. The antibody Capture Level (CL) in resonance units (RU) was monitored. 90 nM RBD (Roche in house, 42 kDa) was injected at 30 μl/min to the captured anti-RBD Antibodies. In another embodiment, the antibodies were injected at 40 μl/min. The analyte association phase was monitored between 3-5 minutes and the dissociation phase was monitored for 5, 10 or 14 minutes. After each measurement cycle the capture systems were regenerated by subsequent injections of 10 mM Glycine buffers pH 2.0 and pH 2.25 at 20 μL/min for 60 seconds.

The binding signatures for the single concentration kinetics were monitored by the BIAcore™ 8K Control-SW V 3.0.11.15423 and evaluated by the BIAcore™ Insight Evaluation SW V 3.0.11.15423, respectively B4000 Control SW V 1.1 and Evaluation SW V 1.1.

Kinetic data were interpreted by report point characterizations and kinetic determinations. Two report points, the recorded signal shortly before the end of the analyte injection, Analyte Binding Late (BL), and the signal shortly before the end of the dissociation time, Stability Late (SL), were used to characterize the antibody/antigen binding stability.

The dissociation rate constant k_{d} (s⁻¹) was calculated according to a Langmuir model and the antibody/antigen complex half-life was calculated in minutes according to the formula t/2 diss=ln(2)/(k_{d}*60).

The Molar Ratio, the binding stoichiometry was calculated by the formula

MR=B (antigen)*MW (antibody)/(MW (antigen)*CL (antibody)).

### Example 3: Kinetic Characterization of SARS-CoV-2 RBD Antibodies

The monoclonal rabbit and mouse RBD antibodies selected by kinetic screening were characterized in further detail.

Measurements were performed using the BIAcore™ 8K and 8K+instruments. RBD concentration series between 0.2-180 nM were injected at flow rates between 30 to 60 μl/min. The association phase was monitored between 3 to 5 minutes, the dissociation phase between 5 to 60 minutes at 37° C.

For the kinetic characterization of the clones 4H10, 1F12, 7G5 and 14F10, the system and sample buffer was as described above, but supplemented with 2 mg/mL Bovine Serum Albumine (BSA). The kinetic rate constants and the dissociation equilibrium constants K_{D} were calculated using a Langmuir 1:1 fit model according to the BIAcore™Insight Evaluation SW V 3.0.11.15423 or using the Langmuir 1:1 fit model from the Scrubber-SW V 2.0c.

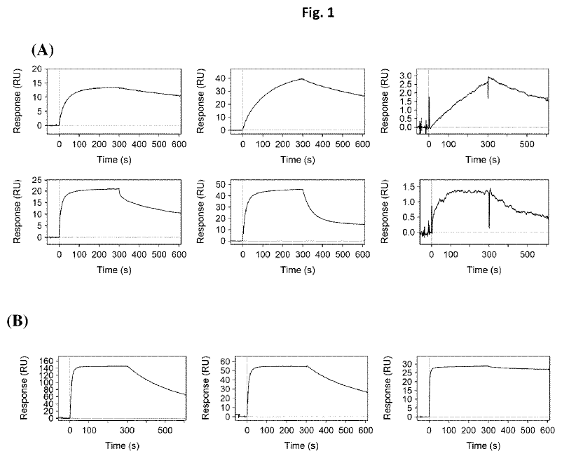

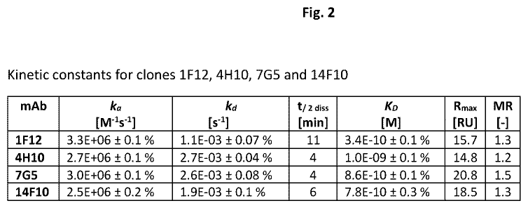

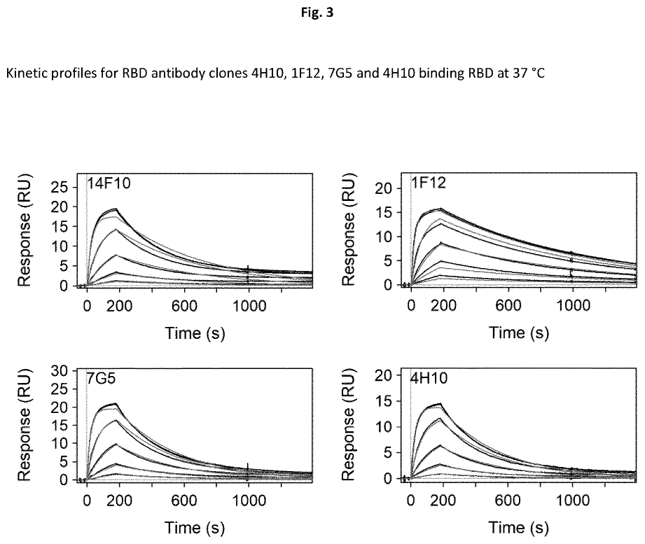

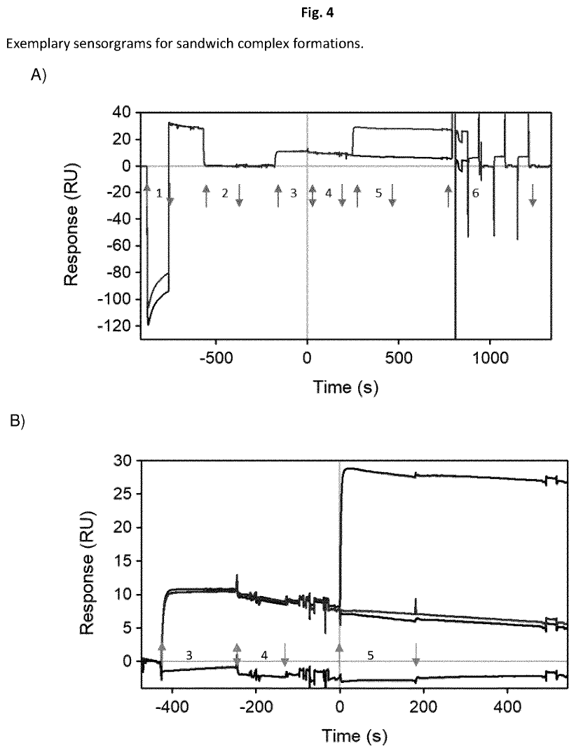

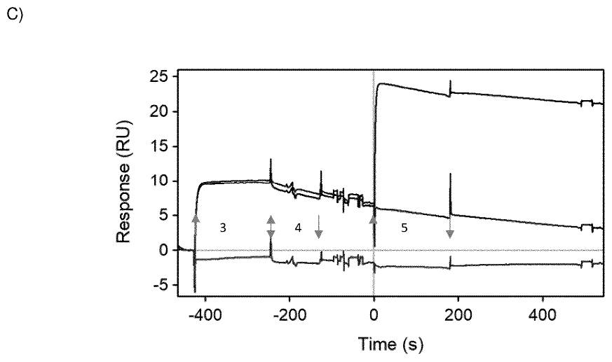

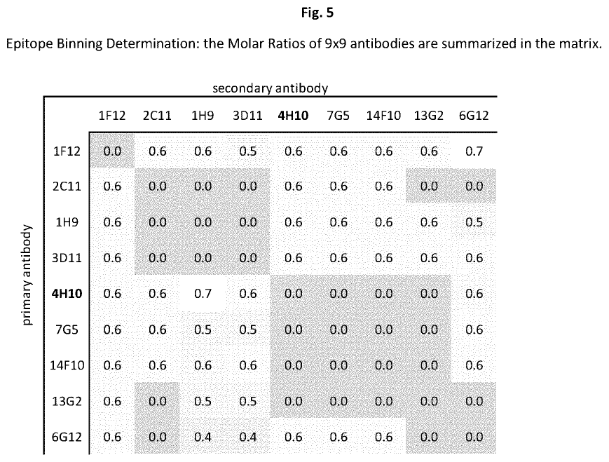

Results of the SPR kinetic screening and characterization of the representative RBD antibodies are shown in FIG. 1, FIG. 2 and FIG. 3, respectively.

All antibodies that met our stringent selection criteria show fast association rates (kₐ) in the range >1.0E+05 M⁻¹s⁻¹ and dissociation rates (k_{d}) below 5.0E-03 s⁻¹. All antibodies display affinities in the nanomolar and subnanomolar range, respectively. FIG. 1 shows examples of antibodies that met the selection criteria as defined above (FIG. 1B) and those antibodies that displayed kinetic signatures that were not suitable for our purposes (FIG. 1A) and therefore deselected with no further investigation. The antibody 1F12 shows high affinity of 0.34 nM±0.1%. Antibody 4H10 displays an affinity to RBD of 1.0 nM±0.1%. The antibodies 7G5 and 14F10 show high affinities with 0.86 nM±0.1% and 0.78 nM±0.3%, respectively (see FIG. 2). Interactions of the antibodies 4H10, 1F12, 7G5 and 14F10 with different concentrations of the RBD (0.2 nM to 13.3 nM) were determined in the presence of BSA at 37° C. Concentration series with a duplicate for concentration 13.3 nM (black) were overlayed by a Langmuir 1:1 fitting model, Rmax global, RI=0 (grey) (FIG. 3).

Similarly the kinetic screening of the generated antibodies was performed using different mutant RBS (variants) using the Bruker SRP-32-Pro instrument. Exemplary results of the SPR kinetic screening and characterization of the clone 1F12 binding to the variants are shown in FIG. 8 to FIG. 12.

Conclusion: As a result of RBD immunization, we generated rabbit and mouse monoclonal IgGs specific for SARS-CoV-2 RBD (wildtype and mutants), but not reacting with the RBD protein from the common cold coronaviruses or MERS (data not shown). This is supported by Biacore SPR and immunoassay analysis results.

In total, 13248 rabbit and 21504 mouse antibodies were pre-screened in a RBD target-specific ELISA. 3427 rabbit and mouse antibodies were tested in SPR experiments. 157 rabbit and mouse RBD antibodies identified via the kinetic screening were further kinetically characterized for binding to the RBD. 63 clones were identified with kinetic properties meeting the criteria for the Elecsys® platform.

### Example 4: Sandwich Complex Formation Experiments

The antibody/antigen sandwich formation experiments were performed at 25° C. on a GE Healthcare BIAcore™ 8K+instrument. A Biacore 2D-PEG-sensor surface was mounted to the instrument and was preconditioned according to the manufacturer's instructions. A rabbit or mouse antibody capture system was utilized as described above. The activation time for the EDC/NHS mixture was 30 seconds. The capture systems were immobilized with up to 400 RU. System and sample buffers were as described above. The system buffer was HBS-ET+pH 7.4 (10 mM HEPES, 150 mM NaCl, 3 mM EDTA, 0.05% (w/v) Tween20, pH 7.4) The system buffer supplemented with 1 mg/mL CMD (Carboxymethyldextran, Fluka) was used as sample buffer. Rabbit or mouse RBD mAbs were tested for sandwich complex formation with RBD protein.

Primary antibody supernatants were diluted and were captured for 2 minutes on each Fc2 sensor channel at 10 μL/min. The capture systems were blocked with 1 μM rabbit normal IgG or a mouse antibody blocking cocktail for 3 minutes at 30 μL/min. Subsequently, 45 nM RBD was injected for 3 minutes. Primary antibody supernatant, diluted 1:20 to 1:50 was repeatedly injected for 2 minutes at 30 μL/min. Secondary antibody solutions were diluted 1:20 to 1:50 and were injected for 3 minutes, followed by 5 minutes dissociation time at 30 μL/min. The system was regenerated as described.

The system was regenerated as described above.

The immune complex stability was evaluated using the SW extension “Epitope Binning” from BIAcore™Insight Evaluation SW V 3.0.11.15423. The sandwich complex formation experiments were interpreted by report points evaluations. Two report points Capture Level (CL), the recorded signal shortly after the end of capturing the primary antibody and the analyte stability early, the recorded signal shortly after the end of the secondary antibody injection, were used to characterize the immune complex stability. The epitope accessibility was quantified as Molar Ratio (MR) by forming a quotient between the resonance units of the secondary antibody binding response signal and the capture level of the primary antibody.

By combining the information from different experiments, 21 distinct RBD epitope regions were identified (data not shown).

### Example 5: Identification of ACE-2 RBD Interface Binders

Anti-RBD antibodies were further investigated for their potential to interfere with the ACE-2/RBD interaction. The experiments were performed at 37° C. on GE Healthcare BIAcore™ 8K+ and 8K instruments. Rabbit mAbs were captured as ligands as described above. RBD and ACE2-FL-His8 (Roche, 87 kDa) were used as analytes in solution and were consecutively injected. 50 nM RBD were injected at 40 μl/min for 3 min and subsequently 250 nM ACE2-FL-His8 were injected at 40 μl/min for 3 minutes, followed by 5 minutes dissociation time.

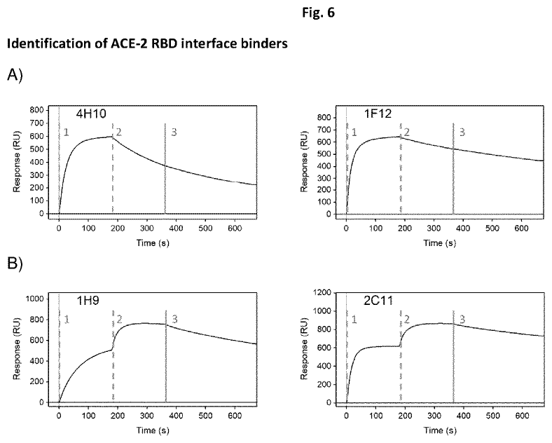

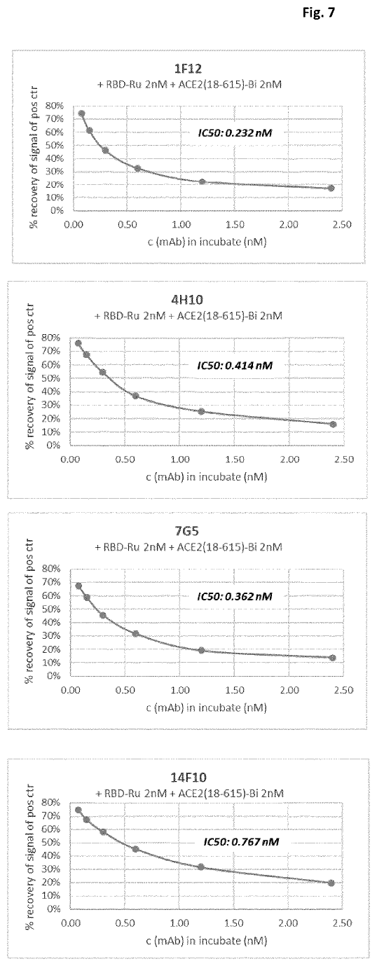

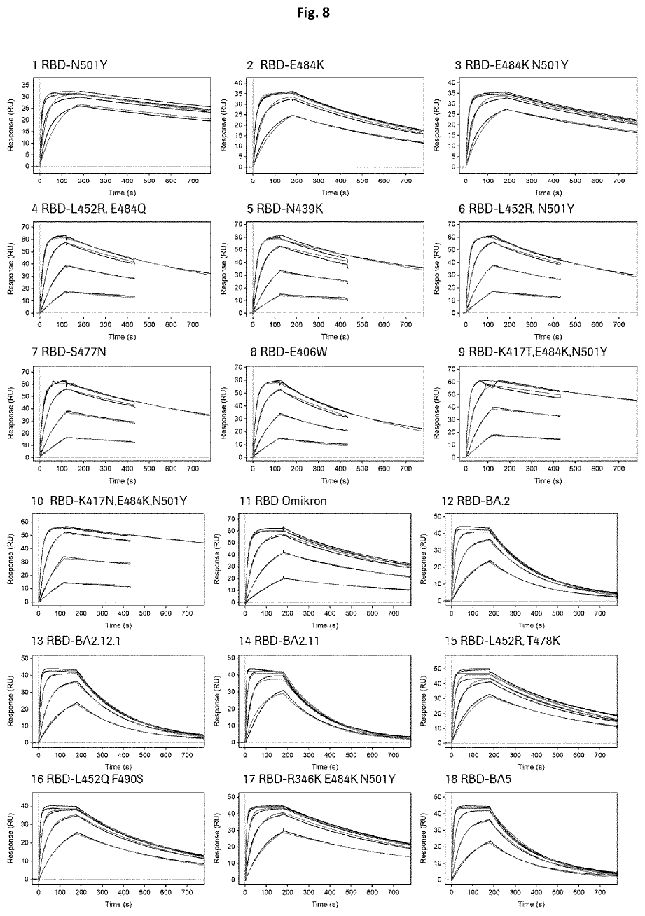

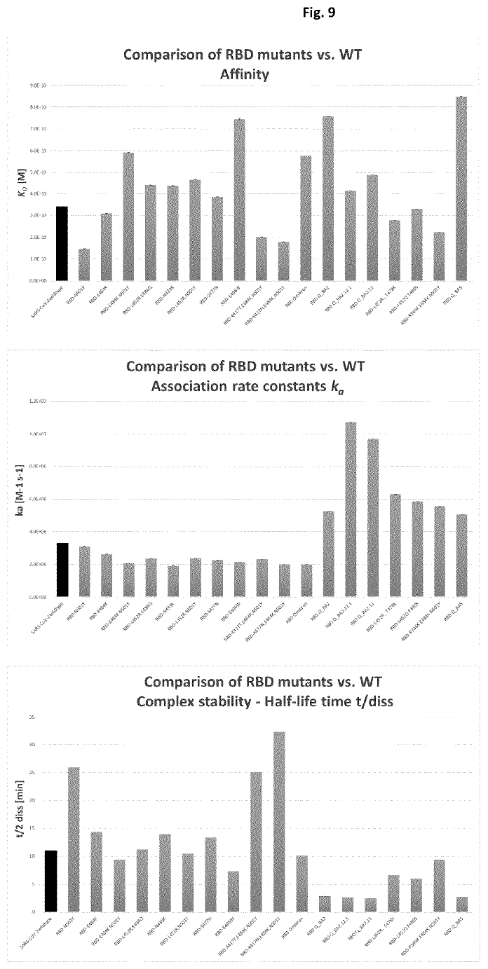

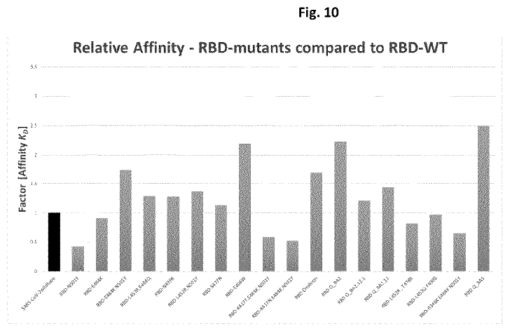

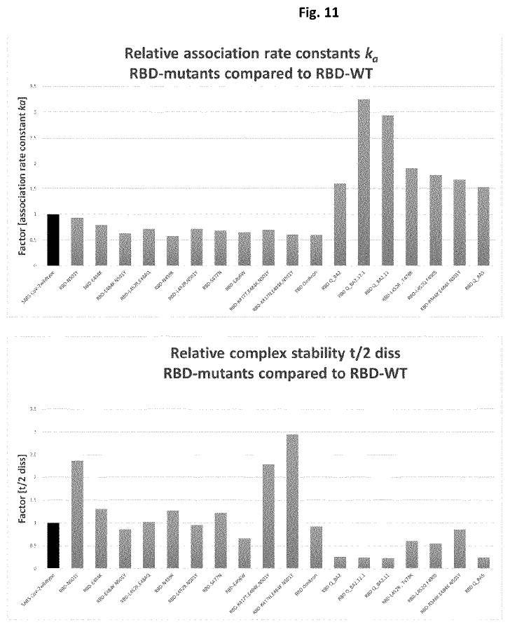

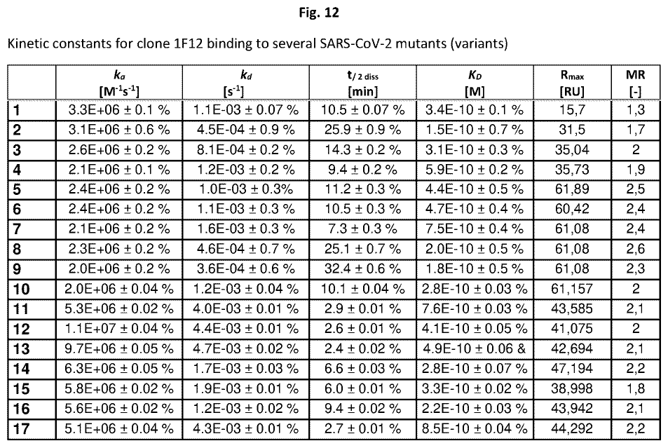

FIG. 6A shows examples of antibodies that bind to the RBD in or close by the ACE-2/RBD interface and completely block the ACE-2/RBD docking.

FIG. 6B shows examples of antibodies that bind remotely from the ACE2/RBD interface.

Hence, a Biacore assay can be used to determine if the antibodies bind in or close by the ACE-2/RBD interface or remotely from the ACE2/RBD interface.

### Example 6: Application in Electrochemiluminescence-Immunoassay (ECLIA)

An ECLIA assay with the RBD antibodies was established to detect antibodies, which are reactive to SARS-CoV-2 spike, and to detect antibodies that bind to wild type RBD and mutants of RBD (data not shown). Monoclonal antibodies (mAbs) that bind to RBD can equally be detected with this assay. As mAbs can be identically reproduced in unlimited quantity and can be quantified with an absolute SI unit (mass per volume), they provide a highly suitable reference calibrator for assay standardization. Capability of such mAbs to interfere with ACE2-RBD binding is of interest, providing an inherent proof that the assay can detect inhibitory antibodies.

To generate information on interference capacity of mAbs with ACE2-RBD binding, we set up a competitive immunoassay on the Elecsys® platform. ACE2 and RBD were labeled to serve as signal specifiers and added at a defined concentration to the assay incubate. The native affinity of ACE2 and RBD caused binding of these molecules that led to signal generation (CLIA method). Baseline reactivity was defined by using the average signal obtained with samples that did not contain RBD specific antibodies (pre-pandemic samples collected before October 2019). No significant difference for signal with negative samples compared to blank sample (diluent) was observed. The mAbs were then added to the reaction, forming a “sample” with defined concentration of RBD specific Ab. The ratio of signal observed with samples containing RBD mAbs and baseline signal served to assess the capacity of the mAb to interfere with ACE2-RBD binding. IC50 was determined by applying regression analyses to serial dilutions of the mAbs.

Exemplary results for mAbs identified to be inhibitory are depicted in FIG. 7.

Assessing inhibition of ACE2-RBD on Elecsys® was compared to inhibition data generated in Biacore measurements. The obtained Elecsys® and Biacore results confirmed each other and the Elecsys® set-up could then be established for automated screening for neutralizing mAbs. Similarly to a Biacore assay, an Elecsys® assay can detect inhibitory/neutralizing antibodies in a patient sample. The results can be used to monitor progression of the disease in patients.

1. An (isolated) monoclonal antibody or antigen-binding fragment thereof that binds to the Receptor Binding Domain (RBD) of the Spike protein of SARS-CoV-2 virus
a) with an association rate constant (kₐ) of more than 2.5E+06 M⁻¹s⁻¹, as determined by surface plasmon resonance,
and/or
b) with a dissociation rate constant (k_{d}) of less than 5.0E-03 s⁻¹, as determined by surface plasmon resonance,
and/or
c) with a half-life time of t_{/2diss} of 4 minutes or more, as determined by surface plasmon resonance,
and/or
d) with a 1:1 or 1:2 stoichiometry.

2. The isolated monoclonal antibody or antigen-binding fragment thereof of claim 1, wherein the antibody is neutralizing.

3. The isolated monoclonal antibody or antigen-binding fragment of claim 1, which
a) comprises CDR-H1, CDR-H2, CDR-H3, CDR-L1, CDR-L2 and CDR-L3 according to SEQ ID NO: 1, 2, 3, 4, 5, and 6, respectively,
b) binds to the same epitope as an antibody comprising CDR-H1, CDR-H2, CDR-H3, CDR-L1, CDR-L2 and CDR-L3 according to SEQ ID NO: 1, 2, 3, 4, 5, and 6, respectively,
or
c) competes for binding to the RBD of the spike protein of SARS-CoV-2 virus with an antibody comprising CDR-H1, CDR-H2, CDR-H3, CDR-L1, CDR-L2 and CDR-L3 according to SEQ ID NO: 1, 2, 3, 4, 5, and 6, respectively.

4. The isolated monoclonal antibody or antigen-binding fragment of claim 3, which
a) comprises FR-H1, FR-H2, FR-H3, FR-H4, FR-L1, FR-L2, FR-L3 and FR-L4 according to SEQ ID NO: 7, 8, 9, 10, 11, 12, 13, and 14, respectively,
b) binds to the same epitope as an antibody comprising FR-H1, FR-H2, FR-H3, FR-H4, FR-L1, FR-L2, FR-L3 and FR-L4 according to SEQ ID NO: 7, 8, 9, 10, 11, 12, 13, and 14, respectively,
or
c) competes for binding to the RBD of the spike protein of SARS-CoV-2 virus with an antibody comprising FR-H1, FR-H2, FR-H3, FR-H4, FR-L1, FR-L2, FR-L3 and FR-L4 according to SEQ ID NO: 7, 8, 9, 10, 11, 12, 13, and 14, respectively.

5. The isolated monoclonal antibody or antigen-binding fragment of claim 1, which
a) comprises CDR-H1, CDR-H2, CDR-H3, CDR-L1, CDR-L2 and CDR-L3 according to SEQ ID NO: 17, 18, 19, 20, 21, and 22, respectively,
b) binds to the same epitope as an antibody comprising CDR-H1, CDR-H2, CDR-H3, CDR-L1, CDR-L2 and CDR-L3 according to SEQ ID NO: 17, 18, 19, 20, 21, and 22, respectively,
or
c) which competes for binding to the RBD of the spike protein of SARS-CoV-2 virus with an antibody comprising CDR-H1, CDR-H2, CDR-H3, CDR-L1, CDR-L2 and CDR-L3 according to SEQ ID NO: 17, 18, 19, 20, 21 and 22, respectively.

6. The isolated monoclonal antibody or antigen-binding fragment of claim 5, which
a) comprises FR-H1, FR-H2, FR-H3, FR-H4, FR-L1, FR-L2, FR-L3, and FR-L4 according to SEQ ID NO: 23, 24, 25, 26, 27, 28, 29, and 30, respectively,
b) binds to the same epitope as an antibody comprising FR-H1, FR-H2, FR-H3, FR-H4, FR-L1, FR-L2, FR-L3, and FR-L4 according to SEQ ID NO: 23, 24, 25, 26, 27, 28, 29, and 30, respectively,
or
c) competes for binding to the RBD of the spike protein of SARS-CoV-2 virus with an antibody comprising FR-H1, FR-H2, FR-H3, FR-H4, FR-L1, FR-L2, FR-L3, and FR-L4 according to SEQ ID NO: 23, 24, 25, 26, 27, 28, 29, and 30, respectively.

7. The isolated monoclonal antibody or antigen-binding fragment of claim 1, which
a) comprises CDR-H1, CDR-H2, CDR-H3, CDR-L1, CDR-L2 and CDR-L3 according to SEQ ID NO: 33, 34, 35, 36, 37, and 38, respectively,
b) binds to the same epitope as an antibody comprising CDR-H1, CDR-H2, CDR-H3, CDR-L1, CDR-L2 and CDR-L3 according to SEQ ID NO: 33, 34, 35, 36, 37, and 38, respectively,
or
c) which competes for binding to the RBD of the spike protein of SARS-CoV-2 virus with an antibody comprising CDR-H1, CDR-H2, CDR-H3, CDR-L1, CDR-L2 and CDR-L3 according to SEQ ID NO: 33, 34, 35, 36, 37, and 38, respectively.

8. The isolated monoclonal antibody or antigen-binding fragment of claim 7, which
a) comprises FR-H1, FR-H2, FR-H3, FR-H4, FR-L1, FR-L2, FR-L3, and FR-L4 according to SEQ ID NO: 39, 40, 41, 42, 43, 44, 45, and 46, respectively,
b) binds to the same epitope as an antibody comprising FR-H1, FR-H2, FR-H3, FR-H4, FR-L1, FR-L2, FR-L3, and FR-L4 according to SEQ ID NO: 39, 40, 41, 42, 43, 44, 45, and 46, respectively,
or
c) competes for binding to the RBD of the spike protein of SARS-CoV-2 virus with an antibody comprising FR-H1, FR-H2, FR-H3, FR-H4, FR-L1, FR-L2, FR-L3, and FR-L4 according to SEQ ID NO: 39, 40, 41, 42, 43, 44, 45, and 46, respectively.

9. The isolated monoclonal antibody or antigen-binding fragment of claim 1, which
a) comprises CDR-H1, CDR-H2, CDR-H3, CDR-L1, CDR-L2 and CDR-L3 according to SEQ ID NO: 49, 50, 51, 52, 53 and 54, respectively,
b) binds to the same epitope as an antibody comprising CDR-H1, CDR-H2, CDR-H3, CDR-L1, CDR-L2 and CDR-L3 according to SEQ ID NO: 49, 50, 51, 52, 53 and 54, respectively,
or
c) which competes for binding to the RBD of the spike protein of SARS-CoV-2 virus with an antibody comprising CDR-H1, CDR-H2, CDR-H3, CDR-L1, CDR-L2 and CDR-L3 according to SEQ ID NO: 49, 50, 51, 52, 53 and 54, respectively.

10. The isolated monoclonal antibody or antigen-binding fragment of claim 9, which
a) comprises FR-H1, FR-H2, FR-H3, FR-H4, FR-L1, FR-L2, FR-L3, and FR-L4 according to SEQ ID NO: 55, 56, 57, 58, 59, 60, 61 and 62, respectively,
b) binds to the same epitope as an antibody comprising FR-H1, FR-H2, FR-H3, FR-H4, FR-L1, FR-L2, FR-L3, and FR-L4 according to SEQ ID NO: 55, 56, 57, 58, 59, 60, 61 and 62, respectively,
or
c) competes for binding to the RBD of the spike protein of SARS-CoV-2 virus with an antibody comprising FR-H1, FR-H2, FR-H3, FR-H4, FR-L1, FR-L2, FR-L3, and FR-L4 according to SEQ ID NO: 55, 56, 57, 58, 59, 60, 61 and 62, respectively.

11. A kit comprising at least one antibody according to claim 1.

12. A nucleic acid encoding an antibody as defined in claim 1.

13. A host cell comprising the nucleic acid of claim 12.

14. A composition comprising the antibody as defined in claim 1.

15. (canceled)
